(19) United States
(12) Patent Application Publication
(10) Pub. No.: US 2021/0106227 A1
(43) Pub. Date: Apr. 15, 2021

(54) SYSTEMS, METHODS, AND DEVICES FOR DETERMINING CARDIAC CONDITION

(71) Applicant: Medtronic, Inc., Minneapolis, MN (US)

(72) Inventors: Subham Ghosh, Blaine, MN (US); Jeffrey M. Gillberg, Coon Rapids, MN (US)

(21) Appl. No.: 17/031,182

(22) Filed: Sep. 24, 2020

Related U.S. Application Data
(60) Provisional application No. 62/913,002, filed on Oct. 9, 2019.

(51) Int. Cl. A61B 5/00; A61B 5/0408

(52) U.S. Cl. CPC A61B 5/0006; A61B 5/0408; A61B 5/6823; A61B 5/0022; A61B 5/002

(57) ABSTRACT

Systems, methods, and devices are described herein for evaluation of patient's cardiac condition based on monitored electrical activity from a plurality of external electrodes. Various information such as electrical heterogeneity information may be generated based on the monitored electrical activity, which may be further analyzed to evaluate the patient's cardiac condition. The systems, methods, and devices may provide an indication of whether the patient may benefit from cardiac therapy based on the evaluation of the patient's cardiac condition.

The present application claims the benefit of U.S. Provisional Application No. 62/913,002, filed Oct. 9, 2019, which is incorporated herein by reference in its entirety.

The disclosure herein relates to systems, methods, and devices for use in determining cardiac conditions of patients to, e.g., identify candidates for cardiac therapy.

Implantable medical devices (IMDs), such as implantable pacemakers, cardioverters, defibrillators, or pacemaker-cardioverter-defibrillators, provide therapeutic electrical stimulation to the heart. IMDs may provide pacing to address bradycardia, or pacing or shocks in order to terminate tachyarrhythmia, such as tachycardia or fibrillation. In some cases, the medical device may sense intrinsic depolarizations of the heart, detect arrhythmia based on the intrinsic depolarizations (or absence thereof), and control delivery of electrical stimulation to the heart if arrhythmia is detected based on the intrinsic depolarizations.

IMDs may also provide cardiac resynchronization therapy (CRT), which is a form of pacing. CRT involves the delivery of pacing to the left ventricle, or both the left and right ventricles. The timing and location of the delivery of pacing pulses to the ventricle(s) may be selected to improve the coordination and efficiency of ventricular contraction.

Patients may benefit from cardiac therapy provided by IMDs or similar technology, but it may be challenging to determine who would benefit from such cardiac therapy. Patients may make appointments to see physicians to analyze and review their cardiac health. Such appointments may include monitoring electrical activity from the patient using, e.g., a 12-lead ECG. However, such information may only be representative of a single “snapshot in time” of the patient's cardiac health. In other words, a physician may only analyze and review a small period, or “slice” of time, of the patient's cardiac health. In such small period of time, the patient's cardiac condition may appear healthy or not so as to qualify for cardiac therapy from, e.g., an 1 MB. When the patient returns home from the appointment, the patient's cardiac condition may decrease such that the patient may be assisted by cardiac therapy from, e.g., an 1 MB.

### SUMMARY

The illustrative systems, methods, and devices described herein may be configured to assist a user in evaluating and analyzing a patient's cardiac condition so as to, e.g., determine whether the patient qualifies or would benefit from cardiac therapy. In one or more embodiments, the systems, methods, and devices may be described as being noninvasive. For example, in some embodiments, the systems, methods, and devices may not need, or include, implantable devices such as leads, probes, sensors, catheters, implantable electrodes, etc. to monitor, or acquire, a plurality of cardiac signals from tissue of the patient. Instead, the systems, methods, and devices may use electrical measurements taken noninvasively using, e.g., a plurality of external electrodes attached to tissue (e.g., the skin) of a patient about the patient's torso.

The illustrative systems, methods, and devices may utilize a plurality of external electrodes, or an “ECG belt,” that may be described as a surface mapping tool for device optimization. The plurality of external electrodes may include a couple of rows of electrodes that are applied to the upper torso, wrapping anterior and posterior sections thereof. The illustrative systems, methods, and devices may be used to measure and monitor electrical dyssynchrony in patients without cardiac therapy devices to identify potential candidacy for therapies like CRT. The illustrative systems, methods, and devices may be described as providing ways of identifying electrical heterogeneity and delayed electrical activation in patients who have symptoms of heart failure and ways of identifying potential candidacy for CRT based on the measured parameters (e.g., electrical heterogeneity metrics) from the external electrodes. Further, the illustrative systems, methods, and devices can be also part of an in-home heart failure management accessory for tracking changes in baseline electrical dyssynchrony and sending alerts to a care team when the patient's cardiac dyssynchrony increases over time and crosses a certain threshold.

The illustrative systems, methods, and devices may be configured to measure electrical activity of the patient during intrinsic activation and to generate electrical heterogeneity/electrical dyssynchrony information for diagnostic purposes in a heart failure patient (e.g., patients without an implanted CRT device). The amount of global electrical heterogeneity may be quantified using various metrics such as a standard deviation of activation times (SDAT) and average left ventricular activation times (LVAT). The candidacy for cardiac therapy (e.g., resynchronization and/or left ventricle pacing therapy) may be identified based on such metrics or parameters of electrical heterogeneity. For example, if SDAT is greater than equal to a certain threshold (e.g., 25 milliseconds (ms)) or if LVAT is greater than equal to a certain threshold (e.g., 50 ms), then the patient may be identified as a candidate for cardiac therapy. The illustrative systems, methods, and devices can be part of an in-home heart failure management accessory in patients with or without an implanted device, where the patient can periodically take measurements with the external electrodes at home and such data taken from the external electrodes may be sent to the cloud for processing of metrics, which can be integrated with the patient's electronic medical records or a centralized database for tracking changes in the patient's cardiac condition over time. If the patient's cardiac condition decreases over time (e.g., dyssynchrony increases over time) and crosses a certain threshold, an alert may be sent to the care team.

One illustrative system for use with a remote computing apparatus to noninvasively evaluate a cardiac condition of a patient may include electrode apparatus comprising a plurality of external electrodes to monitor electrical activity from tissue of a patient and a local computing apparatus comprising processing circuitry and a communication interface. The local computing apparatus may be operably coupled to the electrode apparatus and configured to: monitor, using the electrode apparatus, electrical activity from the tissue of the patient; transmit, using the communication interface, the monitored electrical activity or data related to the monitored electrical activity to a remote computing apparatus; and receive, using the communication interface, an indication of the cardiac condition of the patient from the remote computing apparatus in response to the transmission of the monitored electrical activity or data related to the monitored electrical activity.

One illustrative method for use with a remote system to noninvasively evaluate a cardiac condition of a patient may include monitoring electrical activity from the tissue of the patient using a plurality of external electrodes, transmitting the monitored electrical activity or data related to the monitored electrical activity to a remote computing apparatus, and receiving an indication of the cardiac condition of the patient from the remote computing apparatus in response to the transmission of the monitored electrical activity or data related to the monitored electrical activity.

One illustrative system to noninvasively evaluate a cardiac condition of a patient may include electrode apparatus comprising a plurality of external electrodes to monitor electrical activity from tissue of a patient and a computing apparatus comprising processing circuitry and operably coupled to the electrode apparatus. The computing apparatus may be configured to: measure, using the electrode apparatus, electrical activity from the tissue of the patient during a plurality of sessions, and determine an indication of the cardiac condition of the patient based on the electrical activity over the plurality of sessions.

One illustrative method to noninvasively evaluate a cardiac condition of a patient may include measuring, using a plurality of external electrodes, electrical activity from the tissue of the patient during a plurality of sessions and determining an indication of the cardiac condition of the patient based on the electrical activity over the plurality of sessions.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an illustrative system including electrode apparatus, display apparatus, and computing apparatus.

FIGS. 2-3 are diagrams of illustrative external electrode apparatus for measuring torso-surface potentials.

FIG. 4 is a block diagram of an illustrative method of noninvasive evaluation of the patient's cardiac condition.

FIG. 5 is a block diagram of another illustrative method of noninvasive evaluation of the patient's cardiac condition.

FIG. 6 is a graph of electrical heterogeneity information over a plurality of sessions.

FIG. 7 is another graph of electrical heterogeneity information over a plurality of sessions.

FIG. 8 is a diagram of an illustrative system including an illustrative implantable medical device (IMD).

FIG. 9A is a diagram of the illustrative IMD of FIG. 8.

FIG. 9B is a diagram of an enlarged view of a distal end of the electrical lead disposed in the left ventricle of FIG. 9A.

FIG. 10A is a block diagram of an illustrative IMD, e.g., of the systems of FIGS. 8-9.

FIG. 10B is another block diagram of an illustrative IMD (e.g., an implantable pulse generator) circuitry and associated leads employed in the systems of FIGS. 8-9.

### DETAILED DESCRIPTION OF ILLUSTRATIVE EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

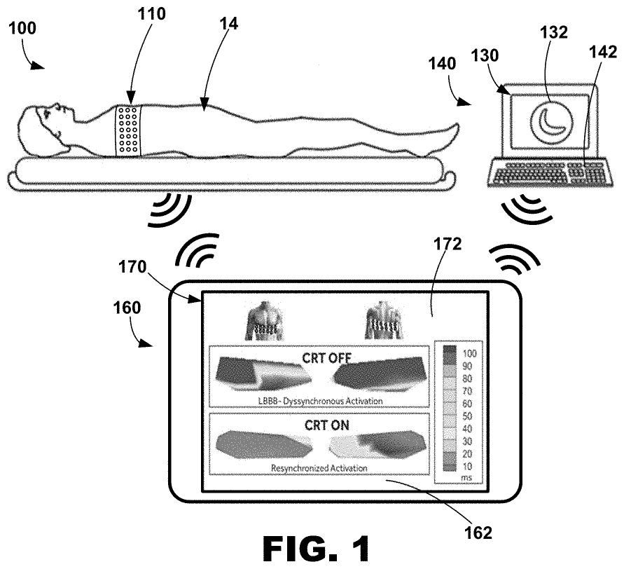

Illustrative systems, methods, and devices shall be described with reference to FIGS. 1-10. It will be apparent to one skilled in the art that elements or processes from one embodiment may be used in combination with elements or processes of the other embodiments, and that the possible embodiments of such systems, methods, and devices using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

A plurality of electrocardiogram (ECG) signals (e.g., torso-surface potentials) may be measured, or monitored, using a plurality of external electrodes positioned about the surface, or skin, of a patient. The ECG signals may be used to evaluate a patient's cardiac condition to, e.g., determine whether or not the patient may benefit from cardiac therapy (e.g., cardiac therapy provided by an implantable medical device performing cardiac resynchronization therapy (CRT)). As described herein, the ECG signals may be gathered or obtained noninvasively since, e.g., implantable electrodes may not be used to measure the ECG signals. Further, the ECG signals may be used to determine cardiac electrical activation times, which may be used to generate various metrics (e.g., electrical heterogeneity information). Such various metrics may then be used to determine the patient's cardiac condition, which may then be used to alert the patient to their cardiac condition, to provide an indication that the patient may benefit from cardiac therapy, to provide additional information to a remote care team, etc.

Various illustrative systems, methods, devices, and graphical user interfaces may be configured to use electrode apparatus including external electrodes, display apparatus, and computing apparatus to noninvasively assist a user (e.g., a physician) in the evaluation of cardiac health and/or determination of the benefit of cardiac therapy. An illustrative system 100 including electrode apparatus 110, remote computing apparatus 140, and a local computing device 160 is depicted in FIG. 1.

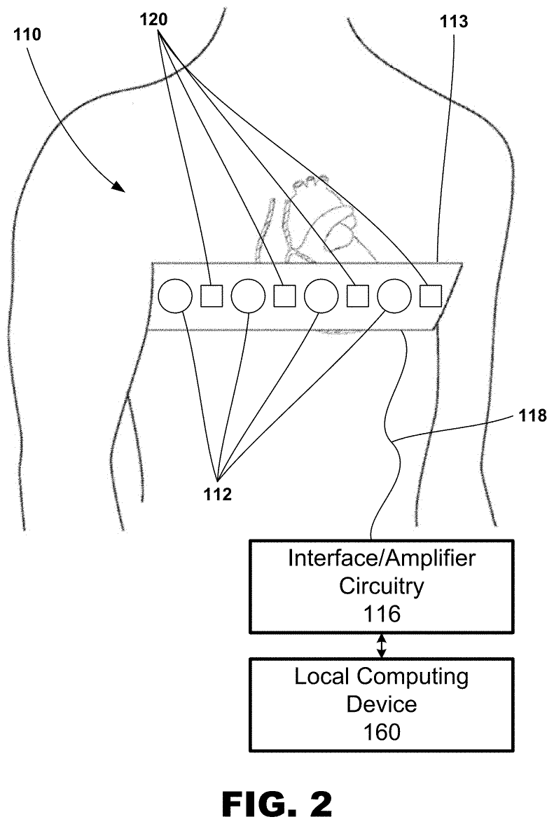

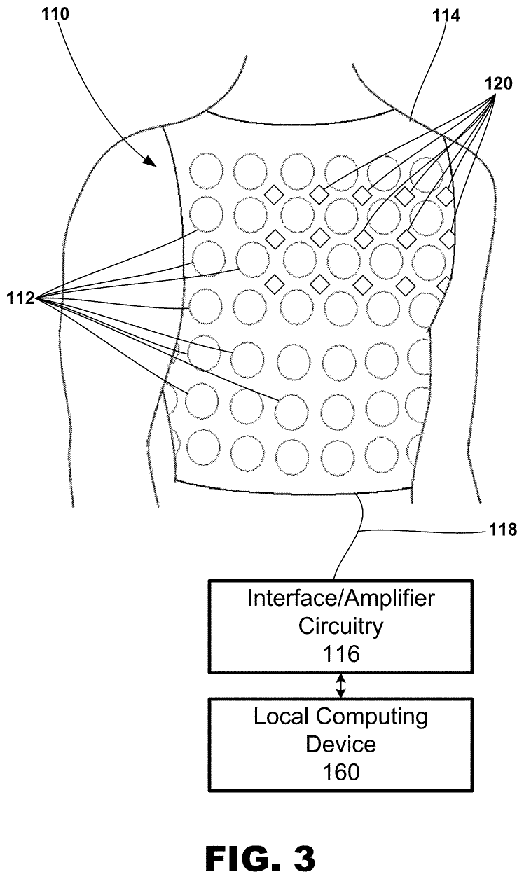

The electrode apparatus 110 as shown includes a plurality of electrodes incorporated, or included, within a band wrapped around the chest, or torso, of a patient 14. The electrode apparatus 110 may be operatively coupled to the local computing device 160 (e.g., through one or wired electrical connections, wirelessly, etc.) to provide electrical signals from each of the electrodes to the local computing apparatus 160 for analysis, evaluation, etc. Illustrative electrode apparatus may be described in U.S. Pat. No. 9,320,446 entitled “Bioelectric Sensor Device and Methods” filed Mar. 27, 2014 and issued on Mar. 26, 2016, which is incorporated herein by reference in its entirety. Further, illustrative electrode apparatus 110 will be described in more detail in reference to FIGS. 2-3.

Although not described herein, the illustrative system 100 may further include imaging apparatus. The imaging apparatus may be any type of imaging apparatus configured to image, or provide images of, at least a portion of the patient in a noninvasive manner. For example, the imaging apparatus may not use any components or parts that may be located within the patient to provide images of the patient except noninvasive tools such as contrast solution. It is to be understood that the illustrative systems, methods, and interfaces described herein may further use imaging apparatus to provide noninvasive assistance to a user (e.g., a physician) to locate, or place, one or more pacing electrodes proximate the patient's heart in conjunction with the configuration of cardiac therapy.

Systems that may be used in conjunction with the illustrative systems, methods, and devices described herein are described in U.S. Pat. App. Pub. No. 2005/0008210 to Evron et al. published on Jan. 13, 2005, U.S. Pat. App. Pub. No. 2006/0074285 to Zarkh et al. published on Apr. 6, 2006, U.S. Pat. No. 8,731,642 to Zarkh et al. issued on May 20, 2014, U.S. Pat. No. 8,861,830 to Brada et al. issued on Oct. 14, 2014, U.S. Pat. No. 6,980,675 to Evron et al. issued on Dec. 27, 2005, U.S. Pat. No. 7,286,866 to Okerlund et al. issued on Oct. 23, 2007, U.S. Pat. No. 7,308,297 to Reddy et al. issued on Dec. 11, 2011, U.S. Pat. No. 7,308,299 to Burrell et al. issued on Dec. 11, 2011, U.S. Pat. No. 7,321,677 to Evron et al. issued on Jan. 22, 2008, U.S. Pat. No. 7,346,381 to Okerlund et al. issued on Mar. 18, 2008, U.S. Pat. No. 7,454,248 to Burrell et al. issued on Nov. 18, 2008, U.S. Pat. No. 7,499,743 to Vass et al. issued on Mar. 3, 2009, U.S. Pat. No. 7,565,190 to Okerlund et al. issued on Jul. 21, 2009, U.S. Pat. No. 7,587,074 to Zarkh et al. issued on Sep. 8, 2009, U.S. Pat. No. 7,599,730 to Hunter et al. issued on Oct. 6, 2009, U.S. Pat. No. 7,613,500 to Vass et al. issued on Nov. 3, 2009, U.S. Pat. No. 7,742,629 to Zarkh et al. issued on Jun. 22, 2010, U.S. Pat. No. 7,747,047 to Okerlund et al. issued on Jun. 29, 2010, U.S. Pat. No. 7,778,685 to Evron et al. issued on Aug. 17, 2010, U.S. Pat. No. 7,778,686 to Vass et al. issued on Aug. 17, 2010, U.S. Pat. No. 7,813,785 to Okerlund et al. issued on Oct. 12, 2010, U.S. Pat. No. 7,996,063 to Vass et al. issued on Aug. 9, 2011, U.S. Pat. No. 8,060,185 to Hunter et al. issued on Nov. 15, 2011, and U.S. Pat. No. 8,401,616 to Verard et al. issued on Mar. 19, 2013, each of which is incorporated herein by reference in its entirety.

The remote computing apparatus 140 and the local computing device 160 may each include display apparatus 130, 160, respectively, that may be configured to display and analyze data such as, e.g., electrical signals (e.g., electrocardiogram data), electrical activation times, electrical heterogeneity information, indications regarding the patient's cardiac condition, determinations of whether the patient may benefit from cardiac therapy, etc. For example, one cardiac cycle, or one heartbeat, of a plurality of cardiac cycles, or heartbeats, represented by the electrical signals collected or monitored by the electrode apparatus 110 may be analyzed and evaluated by one or both of the remote computing apparatus 140 and the local computing device 160 for one or more metrics including activation times and electrical heterogeneity information that may be pertinent to the determination of the patient's cardiac condition and indication of cardiac therapy benefit. More specifically, for example, the QRS complex of a single cardiac cycle may be evaluated for one or more metrics such as, e.g., QRS onset, QRS offset, QRS peak, electrical activation times referenced to the earliest activation time, electrical heterogeneity information (EHI) such as left ventricular or thoracic standard deviation of electrical activation times (LVED), standard deviation of activation times (SDAT), average left ventricular or thoracic surrogate electrical activation times (LVAT), QRS duration (e.g., interval between QRS onset to QRS offset), differences between average left surrogate and average right surrogate activation times, relative or absolute QRS morphology, difference between a higher percentile and a lower percentile of activation times (higher percentile may be 90%, 80%, 75%, 70%, etc. and lower percentile may be 10%, 15%, 20%, 25% and 30%, etc.), other statistical measures of central tendency (e.g., median or mode), dispersion (e.g., mean deviation, standard deviation, variance, interquartile deviations, range), etc. Further, each of the one or more metrics may be location specific. For example, some metrics may be computed from signals recorded, or monitored, from electrodes positioned about a selected area of the patient such as, e.g., the left side of the patient, the right side of the patient, etc.

In at least one embodiment, one or both of the remote computing apparatus 140 and the local computing device 160 may be a server, a personal computer, smartphone, or a tablet computer. The remote computing apparatus 140 may be configured to receive input from input apparatus 142 (e.g., a keyboard) and transmit output to the display apparatus 130, and the local computing device 160 may be configured to receive input from input apparatus 162 (e.g., a touchscreen) and transmit output to the display apparatus 170. One or both of the remote computing apparatus 140 and the local computing device 160 may include data storage that may allow for access to processing programs or routines and/or one or more other types of data, e.g., for analyzing a plurality of electrical signals captured by the electrode apparatus 110, for determining a patient's cardiac condition, for determining whether a patient would qualify as a candidate for cardiac therapy, for determining EHI, for determining QRS onsets, QRS offsets, medians, modes, averages, peaks or maximum values, valleys or minimum values, for determining electrical activation times, for driving a graphical user interface configured to noninvasively assist a user in determining a patient's cardiac condition and whether the patient may benefit from cardiac therapy, etc.

The remote computing apparatus 140 may be operatively coupled to the input apparatus 142 and the display apparatus 130 to, e.g., transmit data to and from each of the input apparatus 142 and the display apparatus 130, and the local computing device 160 may be operatively coupled to the input apparatus 162 and the display apparatus 170 to, e.g., transmit data to and from each of the input apparatus 162 and the display apparatus 170. For example, the remote computing apparatus 140 and the local computing device 160 may be electrically coupled to the input apparatus 142, 162 and the display apparatus 130, 170 using, e.g., analog electrical connections, digital electrical connections, wireless connections, bus-based connections, network-based connections, internet-based connections, etc. As described further herein, a user may provide input to the input apparatus 142, 162 to view and/or select one or more pieces of configuration information related to the cardiac therapy delivered by cardiac therapy apparatus such as, e.g., an implantable medical device.

Each of the remote computing apparatus 140 and local computing device may include a communication interface. The communication interface of the remote computing apparatus 140 may be referred to as the remote communication interface, and the communication interface of the local computing device 160 may be referred to as the local communication interface. The communication interfaces of the remote computing apparatus 140 and the local computing device 160 may be used to communicate with other devices and apparatus such as the electrode apparatus 110 and each other. In one embodiment, the communication interfaces may include a transceiver and antenna for wirelessly communicating with an external device using radio frequency (RF) communication or other communication protocols. Further, the communication interfaces may be configured to be unidirectional or bi-directional.

Although as depicted the input apparatus 142 is a keyboard and the input apparatus 162 is a touchscreen, it is to be understood that the input apparatus 142, 162 may include any apparatus capable of providing input to the remote computing apparatus 140 and the computing device 160 to perform the functionality, methods, and/or logic described herein. For example, the input apparatus 142, 162 may include a keyboard, a mouse, a trackball, a touchscreen (e.g., capacitive touchscreen, a resistive touchscreen, a multi-touch touchscreen, etc.), etc. Likewise, the display apparatus 130, 170 may include any apparatus capable of displaying information to a user, such as a graphical user interface 132, 172 including electrode status information, cardiac condition information, cardiac therapy benefit information, graphical maps of electrical activation, a plurality of signals for the external electrodes over one or more heartbeats, QRS complexes, various cardiac therapy scenario selection regions, various rankings of cardiac therapy scenarios, various pacing parameters, electrical heterogeneity information (EHI), textual instructions, graphical depictions of anatomy of a human heart, images or graphical depictions of the patient's heart, graphical depictions of locations of one or more electrodes, graphical depictions of a human torso, images or graphical depictions of the patient's torso, graphical depictions or actual images of implanted electrodes and/or leads, etc. Further, the display apparatus 130, 170 may include a liquid crystal display, an organic light-emitting diode screen, a touchscreen, a cathode ray tube display, etc.

The processing programs or routines stored and/or executed by the remote computing apparatus 140 and the local computing device 160 may include programs or routines for computational mathematics, matrix mathematics, decomposition algorithms, compression algorithms (e.g., data compression algorithms), calibration algorithms, image construction algorithms, signal processing algorithms (e.g., various filtering algorithms, Fourier transforms, fast Fourier transforms, etc.), standardization algorithms, comparison algorithms, vector mathematics, or any other processing used to implement one or more illustrative methods and/or processes described herein. Data stored and/or used by the remote computing apparatus 140 and the local computing device 160 may include, for example, electrical signal/waveform data from the electrode apparatus 110 (e.g., a plurality of QRS complexes), electrical activation times from the electrode apparatus 110, EHI, cardiac sound/signal/waveform data from acoustic sensors, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein (e.g., electrical signals, electrical heterogeneity information, etc.), or any other data that may be used for carrying out the one and/or more processes or methods described herein.

In one or more embodiments, the illustrative systems, methods, devices, and interfaces may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or methods as described herein or as would be applied in a known fashion.

The one or more programs used to implement the systems, methods, devices, and/or interfaces described herein may be provided using any programmable language, e.g., a high-level procedural and/or object orientated programming language that is suitable for communicating with a computer system. Any such programs may, for example, be stored on any suitable device, e.g., a storage media, that is readable by a general or special purpose program running on a computer system (e.g., including processing apparatus) for configuring and operating the computer system when the suitable device is read for performing the procedures described herein. In other words, at least in one embodiment, the illustrative systems, methods, devices, and interfaces may be implemented using a computer readable storage medium, configured with a computer program, where the storage medium so configured causes the computer to operate in a specific and predefined manner to perform functions described herein. Further, in at least one embodiment, the illustrative systems, methods, devices, and interfaces may be described as being implemented by logic (e.g., object code) encoded in one or more non-transitory media that includes code for execution and, when executed by a processor or processing circuitry, is operable to perform operations such as the methods, processes, and/or functionality described herein.

The remote computing apparatus 140 and the local computing device 160 may be, for example, any fixed or mobile computer system (e.g., a controller, a microcontroller, a personal computer, minicomputer, tablet computer, smartphone, etc.). The exact configurations of the remote computing apparatus 140 and the local computing device 160 are not limiting, and essentially any device including processing circuitry capable of providing suitable computing capabilities and control capabilities (e.g., signal analysis, mathematical functions such as medians, modes, averages, maximum value determination, minimum value determination, slope determination, minimum slope determination, maximum slope determination, graphics processing, etc.) may be used. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by the remote computing apparatus 140 and the local computing device 160 described herein. Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user. The processing circuitry of each of remote computing apparatus 140 and the local computing device 160 may be operably coupled to the communication interface such that the processing circuitry can communication the electrode apparatus 110, each other, and other devices/apparatus.

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes, or programs (e.g., the functionality provided by such systems, processes, or programs) described herein.

The illustrative electrode apparatus 110 may be configured to measure body-surface potentials of a patient 14 and, more particularly, torso-surface potentials of a patient 14. As shown in FIG. 2, the illustrative electrode apparatus 110 may include a set, or array, of external electrodes 112, a strap 113, and interface/amplifier circuitry 116. The electrodes 112 may be attached, or coupled, to the strap 113 and the strap 113 may be configured to be wrapped around the torso of a patient 14 such that the electrodes 112 surround the patient's heart. As further illustrated, the electrodes 112 may be positioned around the circumference of a patient 14, including the posterior, lateral, posterolateral, anterolateral, and anterior locations of the torso of a patient 14.

The illustrative electrode apparatus 110 may be further configured to measure, or monitor, sounds from at least one or both the patient 14. As shown in FIG. 2, the illustrative electrode apparatus 110 may include a set, or array, of acoustic sensors 120 attached, or coupled, to the strap 113. The strap 113 may be configured to be wrapped around the torso of a patient 14 such that the acoustic sensors 120 surround the patient's heart. As further illustrated, the acoustic sensors 120 may be positioned around the circumference of a patient 14, including the posterior, lateral, posterolateral, anterolateral, and anterior locations of the torso of a patient 14.

Further, the electrodes 112 and the acoustic sensors 120 may be electrically connected to interface/amplifier circuitry 116 via wired connection 118. The interface/amplifier circuitry 116 may be configured to amplify the signals from the electrodes 112 and the acoustic sensors 120 and provide the signals to one or both of the remote computing apparatus 140 and the local computing device 160. Other illustrative systems may use a wireless connection to transmit the signals sensed by electrodes 112 and the acoustic sensors 120 to the interface/amplifier circuitry 116 and, in turn, to one or both of the remote computing apparatus 140 and the local computing device 160, e.g., as channels of data. In one or more embodiments, the interface/amplifier circuitry 116 may be electrically coupled to the remote computing apparatus 140 using, e.g., analog electrical connections, digital electrical connections, wireless connections, bus-based connections, network-based connections, internet-based connections, etc.

Although in the example of FIG. 2 the electrode apparatus 110 includes a strap 113, in other examples any of a variety of mechanisms, e.g., tape or adhesives, may be employed to aid in the spacing and placement of electrodes 112 and the acoustic sensors 120. In some examples, the strap 113 may include an elastic band, strip of tape, or cloth. Further, in some examples, the strap 113 may be part of, or integrated with, a piece of clothing such as, e.g., a t-shirt. In other examples, the electrodes 112 and the acoustic sensors 120 may be placed individually on the torso of a patient 14. Further, in other examples, one or both of the electrodes 112 (e.g., arranged in an array) and the acoustic sensors 120 (e.g., also arranged in an array) may be part of, or located within, patches, vests, and/or other manners of securing the electrodes 112 and the acoustic sensors 120 to the torso of the patient 14. Still further, in other examples, one or both of the electrodes 112 and the acoustic sensors 120 may be part of, or located within, two sections of material or two patches. One of the two patches may be located on the anterior side of the torso of the patient 14 (to, e.g., monitor electrical signals representative of the anterior side of the patient's heart, measure surrogate cardiac electrical activation times representative of the anterior side of the patient's heart, monitor or measure sounds of the anterior side of the patient, etc.) and the other patch may be located on the posterior side of the torso of the patient 14 (to, e.g., monitor electrical signals representative of the posterior side of the patient's heart, measure surrogate cardiac electrical activation times representative of the posterior side of the patient's heart, monitor or measure sounds of the posterior side of the patient, etc.). And still further, in other examples, one or both of the electrodes 112 and the acoustic sensors 120 may be arranged in a top row and bottom row that extend from the anterior side of the patient 14 across the left side of the patient 14 to the posterior side of the patient 14.

Yet still further, in other examples, one or both of the electrodes 112 and the acoustic sensors 120 may be arranged in a curve around the armpit area and may have an electrode/sensor-density that less dense on the right thorax that the other remaining areas. For example, the strap 113 can be optimized to form a C-shape for covering areas of torso on the left anterior and left posterior aspects to gather information on left ventricular activation. Further, for example, the strap 113 can be optimized to form a C-shape for application to the right side of the torso to gather information on right ventricular activation. Also, the strap 110 may have a C-shaped design with clearly delineated anatomic markers (e.g., mid-sternal line, left anterior axillary line, posterior vertebral line, etc.) to aid in placing it on the torso.

Additionally, the electrode apparatus 110 can be a reusable belt that's used outside of a medical clinic in follow-up setting or at patient's home. Thus, the electrode apparatus 110 may be used with a patient's smartphone as the local computing device 160 to measure cardiac electrical activity about the patient's torso to be used to, e.g., determine the patient's cardiac condition, determine whether the patient is a candidate for cardiac therapy, etc.

The electrodes 112 may be configured to surround the heart of the patient 14 and record, or monitor, the electrical signals associated with the depolarization and repolarization of the heart after the signals have propagated through the torso of a patient 14. Each of the electrodes 112 may be used in a unipolar configuration to sense the torso-surface potentials that reflect the cardiac signals. The interface/amplifier circuitry 116 may also be coupled to a return or indifferent electrode (not shown) that may be used in combination with each electrode 112 for unipolar sensing.

In some examples, there may be about 12 to about 50 electrodes 112 and about 12 to about 50 acoustic sensors 120 spatially distributed around the torso of a patient. Other configurations may have more or fewer electrodes 112 and more or fewer acoustic sensors 120. It is to be understood that the electrodes 112 and acoustic sensors 120 may not be arranged or distributed in an array extending all the way around or completely around the patient 14. Instead, the electrodes 112 and acoustic sensors 120 may be arranged in an array that extends only part of the way or partially around the patient 14. For example, the electrodes 112 and acoustic sensors 120 may be distributed on the anterior, posterior, and left sides of the patient with less or no electrodes and acoustic sensors proximate the right side (including posterior and anterior regions of the right side of the patient).

One or both of the local computing device 160 and the remote computing apparatus 140 may record and analyze the torso-surface potential signals sensed by electrodes 112 and the sound signals sensed by the acoustic sensors 120, which are amplified/conditioned by the interface/amplifier circuitry 116. One or both of the local computing device 160 and the remote computing apparatus 140 may be configured to analyze the electrical signals from the electrodes 112 to provide electrocardiogram (ECG) signals, information, or data from the patient's heart as will be further described herein. One or both of the local computing device 160 and the remote computing apparatus 140 may be configured to analyze the electrical signals from the acoustic sensors 120 to provide sound signals, information, or data from the patient's body.

Additionally, the remote computing apparatus 140 and the local computing device 160 may be configured to provide graphical user interfaces 132, 172 depicting various information related to the electrode apparatus 110 and the data gathered, or sensed, using the electrode apparatus 110. For example, the graphical user interfaces 132, 172 may depict ECGs including QRS complexes obtained using the electrode apparatus 110 and sound data including sound waves obtained using the acoustic sensors 120 as well as other information related thereto. Illustrative systems, devices, and methods may noninvasively use the electrical information collected using the electrode apparatus 110 and the sound information collected using the acoustic sensors 120 to evaluate a patient's cardiac health and to determine whether cardiac therapy may be beneficial for the patient.

Further, the electrode apparatus 110 may further include reference electrodes and/or drive electrodes to be, e.g. positioned about the lower torso of the patient 14, that may be further used by the system 100. For example, the electrode apparatus 110 may include three reference electrodes, and the signals from the three reference electrodes may be combined to provide a reference signal. Further, the electrode apparatus 110 may use of three caudal reference electrodes (e.g., instead of standard references used in a Wilson Central Terminal) to get a “true” unipolar signal with less noise from averaging three caudally located reference signals.

FIG. 3 illustrates another illustrative electrode apparatus 110 that includes a plurality of electrodes 112 configured to surround the heart of the patient 14 and record, or monitor, the electrical signals associated with the depolarization and repolarization of the heart after the signals have propagated through the torso of the patient 14 and a plurality of acoustic sensors 120 configured to surround the heart of the patient 14 and record, or monitor, the sound signals associated with the heart after the signals have propagated through the torso of the patient 14. The electrode apparatus 110 may include a vest 114 upon which the plurality of electrodes 112 and the plurality of acoustic sensors 120 may be attached, or to which the electrodes 112 and the acoustic sensors 120 may be coupled. In at least one embodiment, the plurality, or array, of electrodes 112 may be used to collect electrical information such as, e.g., surrogate electrical activation times. Similar to the electrode apparatus 110 of FIG. 2, the electrode apparatus 110 of FIG. 3 may include interface/amplifier circuitry 116 electrically coupled to each of the electrodes 112 and the acoustic sensors 120 through a wired connection 118 and be configured to transmit signals from the electrodes 112 and the acoustic sensors 120 to remote computing apparatus 140. As illustrated, the electrodes 112 and the acoustic sensors 120 may be distributed over the torso of a patient 14, including, for example, the posterior, lateral, posterolateral, anterolateral, and anterior locations of the torso of a patient 14.

The vest 114 may be formed of fabric with the electrodes 112 and the acoustic sensors 120 attached to the fabric. The vest 114 may be configured to maintain the position and spacing of electrodes 112 and the acoustic sensors 120 on the torso of the patient 14. Further, the vest 114 may be marked to assist in determining the location of the electrodes 112 and the acoustic sensors 120 on the surface of the torso of the patient 14. In some examples, there may be about 25 to about 256 electrodes 112 and about 25 to about 256 acoustic sensors 120 distributed around the torso of the patient 14, though other configurations may have more or fewer electrodes 112 and more or fewer acoustic sensors 120.

The illustrative systems, methods, and devices may be used to provide noninvasive assistance to a user in the evaluation of a patient's cardiac health and/or evaluation whether the patient may benefit from cardiac therapy. For example, the illustrative systems, methods, and devices may be used to assist a user acquiring cardiac electrical activity in-home and analyzing the acquired cardiac electrical activity to determine the patient's cardiac condition and/or determine whether the patient may benefit from cardiac therapy.

Further, it is to be understood that the remote computing apparatus 140 and the local computing device 160 may be operatively coupled to each other in a plurality of different ways using their respective communication interfaces so as to perform, or execute, the functionality described herein. For example, in the embodiment depicted, the computing device 140 may be wireless operably coupled to the local computing device 160 as depicted by the wireless signal lines emanating therebetween. Additionally, as opposed to wireless connections, one or more of the remote computing apparatus 140 and the remoting computing device 160 may be operably coupled through one or wired electrical connections.

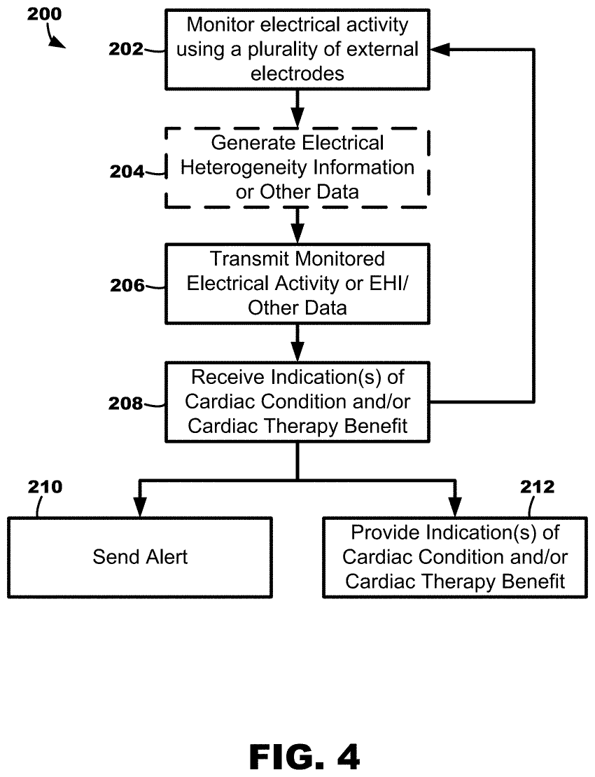

An illustrative method 200 of evaluation of the cardiac condition of a patient is depicted in FIG. 4. The illustrative method 200 may be generally described to be used in the noninvasive evaluation a patient's natural, intrinsic cardiac condition so as to be able to assist in determining whether the patient may benefit from cardiac therapy (e.g., provided by an IMD). The illustrative method 200 may be described as being noninvasive because the method does not use invasive apparatus to perform the evaluation of the patient's cardiac condition. In contrast, cardiac therapy that may be provided or delivered, for example, after it is determined that the patient may benefit from cardiac therapy, may be described as being invasive such as when, e.g., one or more pacing electrodes are implanted proximate a patient's heart. Thus, the illustrative method 200 may be described as being used prior to and without any such invasive cardiac therapy.

The illustrative method 200 may be generally described as an in-home, non-clinical process by which a patient may have their cardiac condition evaluated. In this way, the patient may be able to periodically monitor their cardiac health on more frequent basis than may be possible through a clinic or hospital setting. For example, a patient may be able to perform the method 200 every day or multiple times a day without leaving the comfort and convenience of their own home. Each of these measurement and evaluation performances may be referred to herein as a “session.” Thus, in other words, a patient may perform a session every day or multiple sessions a day without leaving the comfort and convenience of their own home. Additionally, as will be further described herein, the sessions may be tracked, or monitored, over time to provide trends of the patient's cardiac condition, which may be useful in determining whether the patient is a candidate for cardiac therapy.

In this example, the method 200 may be performed solely by a local computing device such as, e.g., a smartphone. It is to be understood that, in other examples, one or more processes of the method 200 may be performed on other devices or apparatus other than the local computing device such as, e.g., a remote computing apparatus.

The method 200 may include monitoring, or measuring, electrical activity using a plurality of external electrodes 202. The plurality of external electrodes may be similar to the external electrodes provided by the electrode apparatus 110 as described herein with respect to FIGS. 1-3. For example, the plurality of external electrodes may be part, or incorporated into, a vest or band that is located about a patient's torso. More specifically, the plurality of electrodes may be described as being surface electrodes positioned in an array configured to be located proximate the skin of the torso of a patient. During process 202, the electrical activity monitored may be without the delivery of cardiac therapy to the patient, and thus, may be referred to as “baseline” or intrinsic electrical activity because no therapy is delivered to the patient such that the patient's heart is in its natural, or intrinsic, rhythm.

In one example, a patient may apply the electrode apparatus to their own torso at home and may initiate the measuring of the electrical activity using their smartphone, i.e., the local computing device. The smartphone may receive and store the electrical activity for the particular session (or multiple previous sessions).

The illustrative method 200 may then optionally generate electrical heterogeneity information (EHI) or other data based on the monitored electrical activity 204. The EHI may be described as information, or data, representative of at least one of mechanical cardiac functionality and electrical cardiac functionality. The EHI and other cardiac therapy information may be described in U.S. Provisional Patent Application No. 61/834,133 entitled “METRICS OF ELECTRICAL DYSSYNCHRONY AND ELECTRICAL ACTIVATION PATTERNS FROM SURFACE ECG ELECTRODES” and filed on Jun. 12, 2013, which is hereby incorporated by reference it its entirety.

Electrical heterogeneity information (e.g., data) may be defined as information indicative of at least one of mechanical synchrony or dyssynchrony of the heart and/or electrical synchrony or dyssynchrony of the heart. In other words, electrical heterogeneity information may represent a surrogate of actual mechanical and/or electrical functionality of a patient's heart. In at least one embodiment, relative changes in electrical heterogeneity information (e.g., from baseline heterogeneity information to therapy heterogeneity information, from a first set of heterogeneity information to a second set of therapy heterogeneity information, etc.) may be used to determine a surrogate value representative of the changes in hemodynamic response (e.g., acute changes in LV pressure gradients). The left ventricular pressure may be typically monitored invasively with a pressure sensor located in the left ventricular of a patient's heart. As such, the use of electrical heterogeneity information to determine a surrogate value representative of the left ventricular pressure may avoid invasive monitoring using a left ventricular pressure sensor.

In at least one embodiment, the electrical heterogeneity information may include a standard deviation of ventricular activation times measured using some or all of the external electrodes, e.g., of the electrode apparatus 110. Further, local, or regional, electrical heterogeneity information may include standard deviations and/or averages of activation times measured using electrodes located in certain anatomic areas of the torso. For example, external electrodes on the left side of the torso of a patient may be used to compute local, or regional, left electrical heterogeneity information.

The electrical heterogeneity information may be generated using one or more various systems and/or methods. For example, electrical heterogeneity information may be generated using an array, or a plurality, of surface electrodes and/or imaging systems as described in U.S. Pat. App. Pub. No. 2012/0283587 A1 published Nov. 8, 2012 and entitled “ASSESSING INTRA-CARDIAC ACTIVATION PATTERNS AND ELECTRICAL DYSSYNCHRONY,” U.S. Pat. App. Pub. No. 2012/0284003 A1 published Nov. 8, 2012 and entitled “ASSESSING INTRA-CARDIAC ACTIVATION PATTERNS”, and U.S. Pat. No. 8,180,428 B2 issued May 15, 2012 and entitled “METHODS AND SYSTEMS FOR USE IN SELECTING CARDIAC PACING SITES,” each of which is incorporated herein by reference in its entirety.

Electrical heterogeneity information may include one or more metrics or indices. For example, one of the metrics, or indices, of electrical heterogeneity may be a standard deviation of activation times (SDAT) measured using some or all of the electrodes on the surface of the torso of a patient. In some examples, the SDAT may be calculated using the estimated cardiac activation times over the surface of a model heart.

Another metric, or index, of electrical heterogeneity may be a left standard deviation of surrogate electrical activation times (LVED) monitored by external electrodes located proximate the left side of a patient. Further, another metric, or index, of electrical heterogeneity may include an average of surrogate electrical activation times (LVAT) monitored by external electrodes located proximate the left side of a patient. The LVED and LVAT may be determined (e.g., calculated, computed, etc.) from electrical activity measured only by electrodes proximate the left side of the patient, which may be referred to as “left” electrodes. The left electrodes may be defined as any surface electrodes located proximate the left ventricle, which includes region to left of the patient's sternum and spine. In one embodiment, the left electrodes may include all anterior electrodes on the left of the sternum and all posterior electrodes to the left of the spine. In another embodiment, the left electrodes may include all anterior electrodes on the left of the sternum and all posterior electrodes. In yet another embodiment, the left electrodes may be designated based on the contour of the left and right sides of the heart as determined using imaging apparatus (e.g., x-ray, fluoroscopy, etc.).

Another illustrative metric, or index, of dyssynchrony may be a range of activation times (RAT) that may be computed as the difference between the maximum and the minimum torso-surface or cardiac activation times, e.g., overall, or for a region. The RAT reflects the span of activation times while the SDAT gives an estimate of the dispersion of the activation times from a mean. The SDAT also provides an estimate of the heterogeneity of the activation times, because if activation times are spatially heterogeneous, the individual activation times will be further away from the mean activation time, indicating that one or more regions of heart have been delayed in activation. In some examples, the RAT may be calculated using the estimated cardiac activation times over the surface of a model heart.

Another illustrative metric, or index, of electrical heterogeneity information may include estimates of a percentage of surface electrodes located within a particular region of interest for the torso or heart whose associated activation times are greater than a certain percentile, such as, for example the 70th percentile, of measured QRS complex duration or the determined activation times for surface electrodes. The region of interest may, e.g., be a posterior, left anterior, and/or left-ventricular region. The illustrative metric, or index, may be referred to as a percentage of late activation (PLAT). The PLAT may be described as providing an estimate of percentage of the region of interest, e.g., posterior and left-anterior area associated with the left ventricular area of heart, which activates late. A large value for PLAT may imply delayed activation of a substantial portion of the region, e.g., the left ventricle, and the potential benefit of electrical resynchronization through CRT by pre-exciting the late region, e.g., of left ventricle. In other examples, the PLAT may be determined for other subsets of electrodes in other regions, such as a right anterior region to evaluate delayed activation in the right ventricle. Furthermore, in some examples, the PLAT may be calculated using the estimated cardiac activation times over the surface of a model heart for either the whole heart or for a particular region, e.g., left or right ventricle, of the heart.

In one or more embodiments, the electrical heterogeneity information may include indicators of favorable changes in global cardiac electrical activation such as, e.g., described in Sweeney et al., “Analysis of Ventricular Activation Using Surface Electrocardiography to Predict Left Ventricular Reverse Volumetric Remodeling During Cardiac Resynchronization Therapy,” Circulation, 2010 Feb. 9, 121(5): 626-34 and/or Van Deursen, et al., “Vectorcardiography as a Tool for Easy Optimization of Cardiac Resynchronization Therapy in Canine LBBB Hearts,” Circulation Arrhythmia and Electrophysiology, 2012 Jun. 1, 5(3): 544-52, each of which is incorporated herein by reference in its entirety. Heterogeneity information may also include measurements of improved cardiac mechanical function measured by imaging or other systems to track motion of implanted leads within the heart as, e.g., described in Ryu et al., “Simultaneous Electrical and Mechanical Mapping Using 3D Cardiac Mapping System: Novel Approach for Optimal Cardiac Resynchronization Therapy,” Journal of Cardiovascular Electrophysiology, 2010 February, 21(2): 219-22, Sperzel et al., “Intraoperative Characterization of Interventricular Mechanical Dyssynchrony Using Electroanatomic Mapping System—A Feasibility Study,” Journal of Interventional Cardiac Electrophysiology, 2012 November, 35(2): 189-96, and/or U.S. Pat. App. Pub. No. 2009/0099619 A1 entitled “METHOD FOR OPTIMIZING CRT THERAPY” and published on Apr. 16, 2009, each of which is incorporated herein by reference in its entirety.

As described herein, data other than EHI may be generated. Such other data related to the monitored electrical activity may include heart rate, heart rate variations/variability, standard ECG-based metrics like QRS morphology and QRS duration, PR intervals, QT intervals and metrics of dispersion of repolarization like QT dispersion across the different electrodes in the belt, etc.

Further, the other data related to the monitored electrical activity may further include compressed, condensed, and/or filtered monitored electrical activity. More specifically, the monitored electrical activity may include a large amount of data, and the illustrative local computing device may compress and condense the data as well as remove problematic, erroneous, or extraneous data through various filtering techniques.

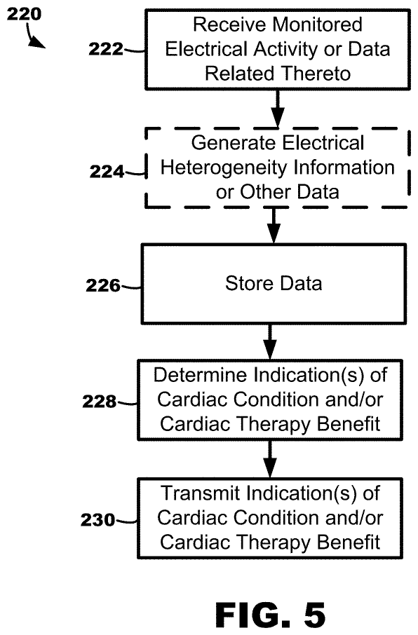

The illustrative method 200 may then transmit the monitored electrical activity or EHI/other data from the local computing device to a remote computing apparatus 206, and subsequently, receive one or more indications regarding the cardiac condition of the patient and whether the patient may benefit from cardiac therapy from the remote computing apparatus 208. The processes for generating such indications regarding the cardiac condition of the patient and whether the patient may benefit from cardiac therapy are described further herein with respect to method 220 depicted in FIG. 5.

The illustrative method 200 may then send an alert 210 to, e.g., an electronic medical records (EMR) system or a care team for the patient if it is determined that the patient's cardiac condition is worsening or that the patient may benefit from cardiac therapy.

Additionally, the illustrative method 200 may provide an indication of the patient's cardiac condition and/or cardiac therapy benefit to the patient 212. Such indications may be provided in various forms such as textual descriptions, numerical scales, etc. on the local computing device such as, e.g., a smartphone. For example, if the patient's cardiac condition is worsening, the indication of the patient's cardiac condition may be “Cardiac condition worsening—please see care provider.” Further, for example, the patient's cardiac condition may be indicated on a range of 0 to 5, with 0 indicating a healthy cardiac condition, 3 indicating a worsening cardiac condition that needs non-urgent care, and a 5 indicating an unhealthy cardiac condition that needs urgent attention. Further, for example, the indication of cardiac therapy benefit may be “Cardiac therapy not needed” if the patient would not benefit from cardiac therapy or “Cardiac therapy may be beneficial—See care provider” if the patient may benefit from cardiac therapy.

Additionally, the method 200 may continue to loop back to monitoring electrical activity 202 after receiving indications(s) of cardiac condition and cardiac therapy benefit 208. Each loop may be considered a single monitoring analysis session. As will be described further herein, the data across multiple sessions may be useful in determining the patient's cardiac condition and whether the patient may benefit from cardiac therapy.

A further illustrative method 220 of evaluation of the cardiac condition of a patient is depicted in FIG. 5. The illustrative method 220 may be generally described as the processes that may occur on a remote computing apparatus upon reception of monitored electrical activity or data related thereto from a local computing device. Generally, the method 220 may analyze the monitored electrical activity or data related thereof and provide indications of cardiac condition and/or benefit from cardiac therapy (e.g., provided by an IMD) back to the local computing device. In this example, the method 220 may be performed solely by a remote computing apparatus such as, e.g., one or more servers. It is to be understood that, in other examples, one or more processes of the method 220 may be performed on other devices or apparatus other than the remote computing apparatus. For instance, one or more (e.g., all) of the processes of method 220 may be performed by the local computing device such that, e.g., no remote computing apparatus may be needed to evaluate a patient's cardiac condition and determine whether the patient may benefit from cardiac therapy.

Similar to method 200, the illustrative method 220 may be described as being noninvasive because the method does not use invasive apparatus to perform the evaluation of the patient's cardiac condition. In contrast, cardiac therapy that may be provided or delivered, for example, after it is determined that the patient may benefit from cardiac therapy, may be described as being invasive such as when, e.g., one or more pacing electrodes are implanted proximate a patient's heart. Thus, the illustrative method 220 may be described as being used to prior to and without any such invasive cardiac therapy. Further, the illustrative method 220 may be generally described as remote data analysis and monitoring of data collected from in-home, non-clinical processes.

The method 220 may first include receiving monitored electrical activity or data related thereto from a local computing device 222. More specifically, for example, the method 200 includes process 206 where monitored electrical activity or data related thereto is sent to a remote computing apparatus, and the method 220 includes process 222 where the monitored electrical activity or data related thereto is received by the remote computing apparatus. As described herein, the local computing device may transmit, or sent, the monitored electrical activity or data related thereto such as, e.g., compressed monitored electrical activity, filtered monitored electrical activity, EHI based on the monitored electrical activity, other computed, or generated, data based on the monitored electrical activity, etc.

Similar to process 204 of method 200, the illustrative method 220 may optionally generate EHI or other data based on the received, monitored electrical activity or data related thereto 224. This process is optional because the method 200 may have already generated the EHI or other data using the local computing device, and subsequently, only transmitted the EHI or other data to the remote computing apparatus. Thus, the remote computing apparatus may not need to generate EHI or other data since, e.g., it may have already been generated and the raw monitored electrical activity may not have been sent from the local computing device.

The method 220 further includes storing data 226. The data may be the received monitored electrical activity or data related thereto such as EHI or other data generated by the local computing device, or EHI or other data generated by the remote computing apparatus in process 224. Regardless, the data may be stored chronologically so as to be able track, or monitor, data related to the patient over time to assist in determining the patient's cardiac condition and/or whether the patient may be benefit from cardiac therapy.

The method 220 further includes determining one or more indications of the patient's cardiac condition and/or cardiac therapy benefit (e.g., whether or not the patient may benefit from cardiac therapy) based on one or more of the received monitored electrical activity or data related thereto, generated EHI or other data by the remote computing apparatus, and stored data 228.

For example, in one embodiment, the method 220 may compare the standard deviation of electrical activation times (SDAT) monitored by the plurality of external electrodes to a SDAT threshold value. The SDAT threshold value may be between about 10 milliseconds and about 45 milliseconds. In at least one embodiment, the SDAT threshold value is 25 milliseconds. If the patient's SDAT is greater than or equal to the SDAT threshold value, then it may be determined that the patient's cardiac condition is worsening and may benefit from cardiac therapy.

Further, for example, in one embodiment, the method 220 may compare the average electrical activation times measured proximate the left side of the patient (LVAT) to a LVAT threshold value. The LVAT threshold value may be between about 35 milliseconds and about 80 milliseconds. In at least one embodiment, the LVAT threshold value is 50 milliseconds. If the patient's LVAT is greater than or equal to the LVAT threshold value, then it may be determined that the patient's cardiac condition is worsening and may benefit from cardiac therapy.

After the indications of the patient's cardiac condition and cardiac therapy benefit have been generated, the method 220 may transmit such indications of the patient's cardiac condition and cardiac therapy benefit from the remote computing apparatus to the local computing device of the patient 230 such that the patient may be informed of such indications.

As described herein, the monitored electrical activity and data related thereto such as EHI may be stored 226 by the remoting computing apparatus. Although not shown, such data may also be stored by the local computing apparatus. The storage of such data may allow the data to be analyzed over time to determine cardiac condition and cardiac therapy benefit indications. For example, process 228 may include tracking the transmitted monitored electrical activity or data related to the monitored electrical activity from the local computing apparatus over a time period and determining a trend in the transmitted monitored electrical activity or data related to the monitored electrical activity over the time period. Such trend could be determined over many sessions or simply two sessions such as an initial session and the most present session or the last session and the present session.

For example, in one embodiment, the method 220 may compare a SDAT percentage increase of the present SDAT from an initial SDAT monitored by the plurality of external electrodes to a SDAT percentage value. The selected SDAT percentage value may be between about 5% and about 20%. In at least one embodiment, the SDAT percentage value is 10%. If the patient's SDAT percentage increase is greater than or equal to the SDAT percentage value, then it may be determined that the patient's cardiac condition is worsening and may benefit from cardiac therapy. In other words, if the standard deviation of electrical activation times for a session has increased by a threshold percentage from an initial session, then it may be determined that the patient's cardiac condition is worsening and may benefit from cardiac therapy.

Further, for example, in one embodiment, the method 220 may compare a LVAT percentage increase of the present LVAT from an initial LVAT monitored by the plurality of external electrodes to a LVAT percentage value. The LVAT percentage value may be between about 5% and about 20%. In at least one embodiment, the selected LVAT percentage value is 10%. If the patient's LVAT percentage increase is greater than or equal to the LVAT percentage value, then it may be determined that the patient's cardiac condition is worsening and may benefit from cardiac therapy. In other words, if the left average of electrical activation times for a session has increased by a percentage from an initial session, then it may be determined that the patient's cardiac condition is worsening and may benefit from cardiac therapy.

Further, for example, in one embodiment, the method 220 may compare the absolute value of the LVAT monitored by the plurality of external electrode to a LVAT threshold value. If the absolute value of the LVAT exceeds the LVAT threshold value for a selected percentage of a plurality of measurements, then it may be determined the patients cardiac dyssynchrony has worsened and may benefit from resynchronization pacing. The threshold may be between about 50 ms and about 70 ms, and the certain percentage may be between about 60% and about 85%. For example, in one embodiment that LVAT threshold value is 60 ms and the certain percentage is 65%. In other words, if the absolute value of the LVAT exceeds the LVAT threshold value for M number of times over the last N measurements, then it may be determined the patients cardiac dyssynchrony has worsened and may benefit from resynchronization pacing. The value of M and N could be 3 and 5, 4 and 6, 5 and 7, 6 and 8, and 8 and 10, respectively

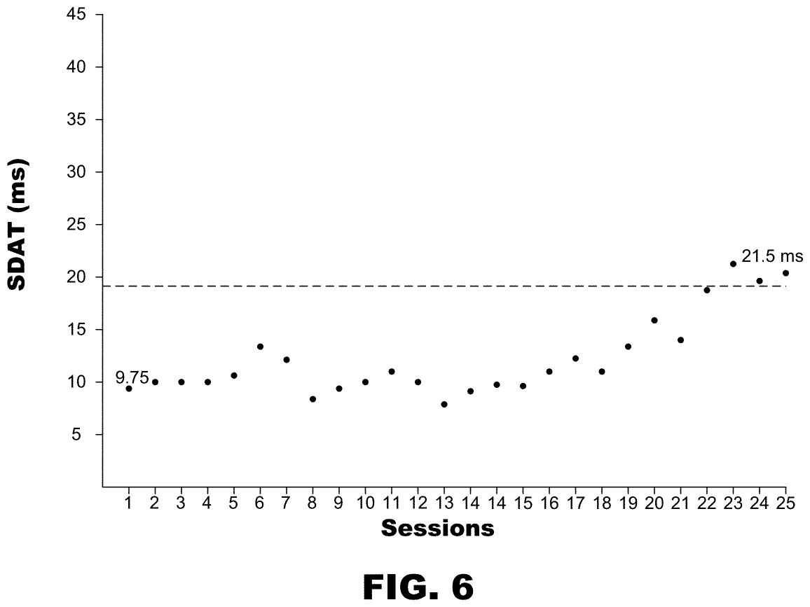

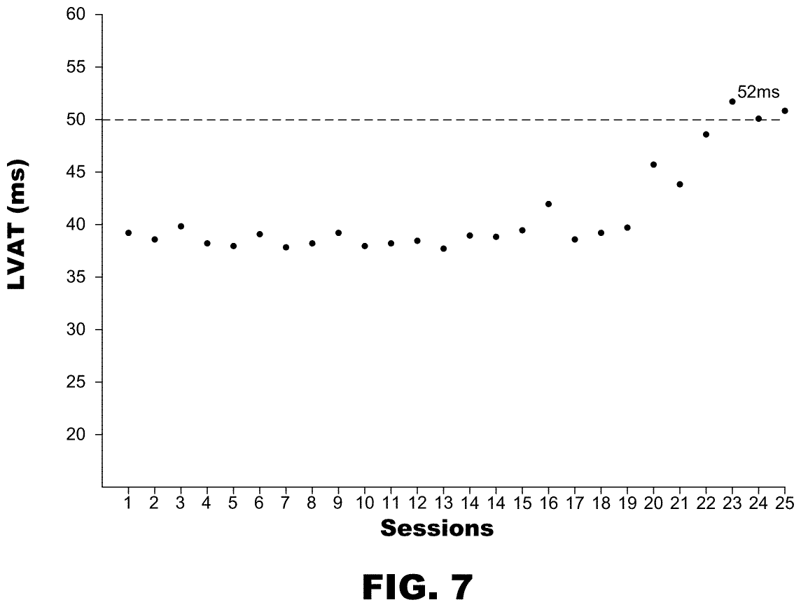

A few illustrative graphs of electrical heterogeneity information over a plurality of sessions are depicted in FIGS. 6-7. More specifically, SDAT is plotted over 25 sessions in FIG. 6 and LVAT is plotted over 25 sessions in FIG. 7. Each session may be recorded daily. Thus, the session may include a 24 duration therebetween.

It is to be understood, however, that sessions may recorded, or monitored, more or less frequently than daily. For example, sessions may be described in terms of the duration therebetween. The duration between sessions may be between about 1 hour and 1 week. In at least one embodiment, the duration between sessions is greater than or equal to 12 hours. The duration between sessions may be greater than or equal to 1 hour, greater than or equal to 2 hours, greater than or equal to 3 hours, greater than or equal to 6 hours, greater than or equal to 12 hours, greater than or equal to 1 day, greater than or equal to 5 days, etc. and/or less than or equal to 10 days, less than or equal to 7 days, less than or equal to 3 days, less than or equal to 2 days, less than or equal to 18 hours, less than or equal to 15 hours, less than or equal to 10 hours, etc.

As shown in FIG. 6, the electrical activity monitored during the initial session generated a SDAT of 9.75 milliseconds. In this example, the SDAT percentage increase threshold is 100% of the initial SDAT or 19.5 milliseconds, which is indicated by the dashed line. As can been seen, the patient's SDAT varies over the about the first sixteen sessions before increasing beyond the 19.5 milliseconds threshold during session 23. Thus, the illustrative systems, methods, and devices may indicate after session 23 that the patient's cardiac condition has worsened and/or that the patient may benefit from cardiac therapy.

In the example depicted in FIG. 7, a fixed LVAT threshold value is used. The LVAT threshold value is 50 milliseconds as indicated by the dashed line. As shown, the patient's LVAT varies over the about the first twenty sessions before increasing to beyond the 50 milliseconds threshold during session 23. Thus, the illustrative systems, methods, and devices may indicate after session 23 that the patient's cardiac condition has worsened and/or that the patient may benefit from cardiac therapy.

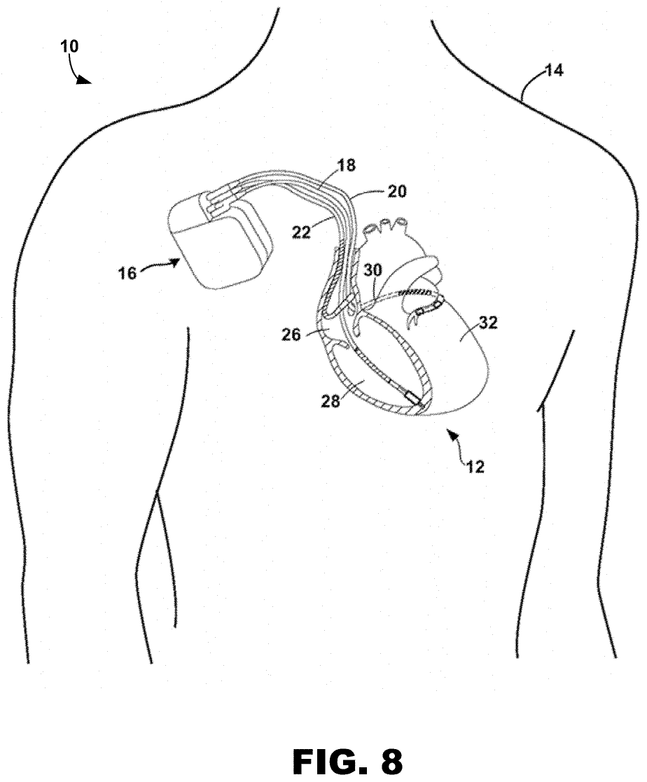

As described herein, the illustrative systems, methods, and devices may provide an indication of whether a patient may benefit from cardiac therapy. Such cardiac therapy systems and devices that may be used by patients (after being instructed by the illustrative systems, methods, and devices that they may be candidates for cardiac therapy) are further described herein with reference to FIGS. 8-10.

FIG. 8 is a conceptual diagram illustrating an illustrative therapy system 10 that may be used to deliver pacing therapy to a patient 14. Patient 14 may, but not necessarily, be a human. The therapy system 10 may include an implantable medical device 16 (IMD), which may be coupled to leads 18, 20, 22. The IMD 16 may be, e.g., an implantable pacemaker, cardioverter, and/or defibrillator, that delivers, or provides, electrical signals (e.g., paces, etc.) to and/or senses electrical signals from the heart 12 of the patient 14 via electrodes coupled to one or more of the leads 18, 20, 22.

The leads 18, 20, 22 extend into the heart 12 of the patient 14 to sense electrical activity of the heart 12 and/or to deliver electrical stimulation to the heart 12. In the example shown in FIG. 8, the right ventricular (RV) lead 18 extends through one or more veins (not shown), the superior vena cava (not shown), and the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins and the vena cava, and into the right atrium 26 of the heart 12.

The IMD 16 may sense, among other things, electrical signals attendant to the depolarization and repolarization of the heart 12 via electrodes coupled to at least one of the leads 18, 20, 22. In some examples, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., A-V delay and other various timings, pulse wide, amplitude, voltage, burst length, etc. Further, the IMD 16 may be operable to use various electrode configurations to deliver pacing therapy, which may be unipolar, bipolar, quadripoloar, or further multipolar. For example, a multipolar lead may include several electrodes that can be used for delivering pacing therapy. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the 1 MB. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode. The IMD 16 may also provide defibrillation therapy and/or cardioversion therapy via electrodes located on at least one of the leads 18, 20, 22. Further, the 1 MB 16 may detect arrhythmia of the heart 12, such as fibrillation of the ventricles 28, 32, and deliver defibrillation therapy to the heart 12 in the form of electrical pulses. In some examples, 1 MB 16 may be programmed to deliver a progression of therapies, e.g., pulses with increasing energy levels, until a fibrillation of heart 12 is stopped.

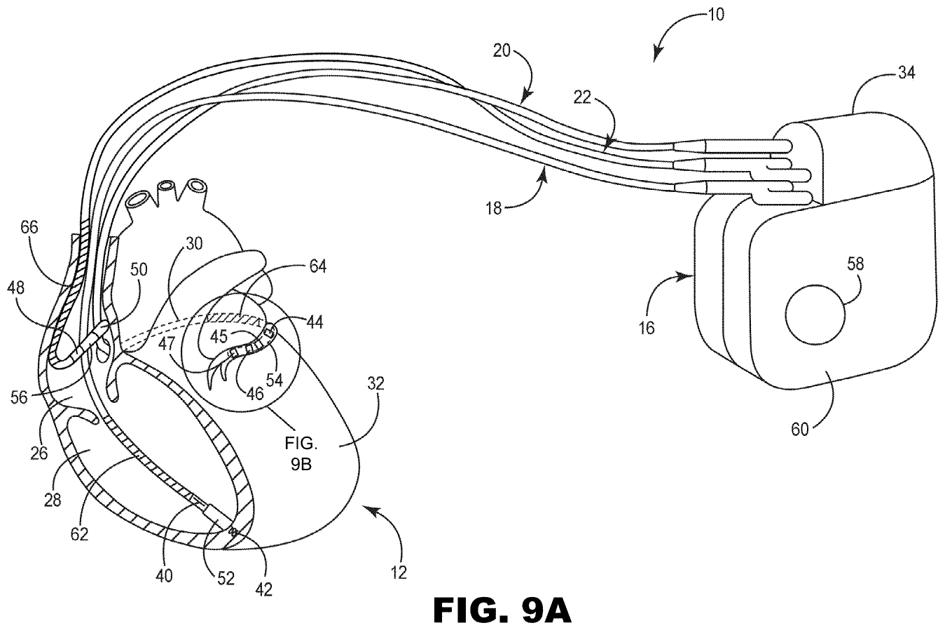

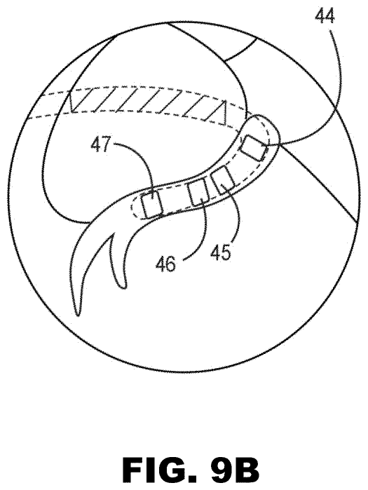

FIGS. 9A-9B are conceptual diagrams illustrating the 1 MB 16 and the leads 18, 20, 22 of therapy system 10 of FIG. 8 in more detail. The leads 18, 20, 22 may be electrically coupled to a therapy delivery module (e.g., for delivery of pacing therapy), a sensing module (e.g., for sensing one or more signals from one or more electrodes), and/or any other modules of the IMD 16 via a connector block 34. In some examples, the proximal ends of the leads 18, 20, 22 may include electrical contacts that electrically couple to respective electrical contacts within the connector block 34 of the 1 MB 16. In addition, in some examples, the leads 18, 20, 22 may be mechanically coupled to the connector block 34 with the aid of set screws, connection pins, or another suitable mechanical coupling mechanism.

Each of the leads 18, 20, 22 includes an elongated insulative lead body, which may carry a number of conductors (e.g., concentric coiled conductors, straight conductors, etc.) separated from one another by insulation (e.g., tubular insulative sheaths). In the illustrated example, bipolar electrodes 40, 42 are located proximate to a distal end of the lead 18. In addition, bipolar electrodes 44, 45, 46, 47 are located proximate to a distal end of the lead 20 and bipolar electrodes 48, 50 are located proximate to a distal end of the lead 22.

The electrodes 40, 44, 45, 46, 47, 48 may take the form of ring electrodes, and the electrodes 42, 50 may take the form of extendable helix tip electrodes mounted retractably within the insulative electrode heads 52, 54, 56, respectively. Each of the electrodes 40, 42, 44, 45, 46, 47, 48, 50 may be electrically coupled to a respective one of the conductors (e.g., coiled and/or straight) within the lead body of its associated lead 18, 20, 22, and thereby coupled to a respective one of the electrical contacts on the proximal end of the leads 18, 20, 22.

Additionally, electrodes 44, 45, 46 and 47 may have an electrode surface area of about 5.3 mm² to about 5.8 mm². Electrodes 44, 45, 46, and 47 may also be referred to as LV1, LV2, LV3, and LV4, respectively. The LV electrodes (i.e., left ventricle electrode 1 (LV1) 44, left ventricle electrode 2 (LV2) 45, left ventricle electrode 3 (LV3) 46, and left ventricle 4 (LV4) 47 etc.) on the lead 20 can be spaced apart at variable distances. For example, electrode 44 may be a distance of, e.g., about 21 millimeters (mm), away from electrode 45, electrodes 45 and 46 may be spaced a distance of, e.g. about 1.3 mm to about 1.5 mm, away from each other, and electrodes 46 and 47 may be spaced a distance of, e.g. 20 mm to about 21 mm, away from each other.

The electrodes 40, 42, 44, 45, 46, 47, 48, 50 may further be used to sense electrical signals (e.g., morphological waveforms within electrograms (EGM)) attendant to the depolarization and repolarization of the heart 12. The electrical signals are conducted to the IMD 16 via the respective leads 18, 20, 22. In some examples, the IMD 16 may also deliver pacing pulses via the electrodes 40, 42, 44, 45, 46, 47, 48, 50 to cause depolarization of cardiac tissue of the patient's heart 12. In some examples, as illustrated in FIG. 9A, the IMD 16 includes one or more housing electrodes, such as housing electrode 58, which may be formed integrally with an outer surface of a housing 60 (e.g., hermetically-sealed housing) of the IMD 16 or otherwise coupled to the housing 60. Any of the electrodes 40, 42, 44, 45, 46, 47, 48, 50 may be used for unipolar sensing or pacing in combination with the housing electrode 58. It is generally understood by those skilled in the art that other electrodes can also be selected to define, or be used for, pacing and sensing vectors. Further, any of electrodes 40, 42, 44, 45, 46, 47, 48, 50, 58, when not being used to deliver pacing therapy, may be used to sense electrical activity during pacing therapy.

As described in further detail with reference to FIG. 9A, the housing 60 may enclose a therapy delivery module that may include a stimulation generator for generating cardiac pacing pulses and defibrillation or cardioversion shocks, as well as a sensing module for monitoring the electrical signals of the patient's heart (e.g., the patient's heart rhythm). The leads 18, 20, 22 may also include elongated electrodes 62, 64, 66, respectively, which may take the form of a coil. The IMD 16 may deliver defibrillation shocks to the heart 12 via any combination of the elongated electrodes 62, 64, 66 and the housing electrode 58. The electrodes 58, 62, 64, 66 may also be used to deliver cardioversion pulses to the heart 12. Further, the electrodes 62, 64, 66 may be fabricated from any suitable electrically conductive material, such as, but not limited to, platinum, platinum alloy, and/or other materials known to be usable in implantable defibrillation electrodes. Since electrodes 62, 64, 66 are not generally configured to deliver pacing therapy, any of electrodes 62, 64, 66 may be used to sense electrical activity and may be used in combination with any of electrodes 40, 42, 44, 45, 46, 47, 48, 50, 58. In at least one embodiment, the RV elongated electrode 62 may be used to sense electrical activity of a patient's heart during the delivery of pacing therapy (e.g., in combination with the housing electrode 58, or defibrillation electrode-to-housing electrode vector).

The configuration of the illustrative therapy system 10 illustrated in FIGS. 8-10 is merely one example. In other examples, the therapy system may include epicardial leads and/or patch electrodes instead of or in addition to the transvenous leads 18, 20, 22 illustrated in FIG. 8. Additionally, in other examples, the therapy system 10 may be implanted in/around the cardiac space without transvenous leads (e.g., leadless/wireless pacing systems) or with leads implanted (e.g., implanted transvenously or using approaches) into the left chambers of the heart (in addition to or replacing the transvenous leads placed into the right chambers of the heart as illustrated in FIG. 8). Further, in one or more embodiments, the IMD 16 need not be implanted within the patient 14. For example, the IMD 16 may deliver various cardiac therapies to the heart 12 via percutaneous leads that extend through the skin of the patient 14 to a variety of positions within or outside of the heart 12. In one or more embodiments, the system 10 may utilize wireless pacing (e.g., using energy transmission to the intracardiac pacing component(s) via ultrasound, inductive coupling, RF, etc.) and sensing cardiac activation using electrodes on the can/housing and/or on subcutaneous leads.

In other examples of therapy systems that provide electrical stimulation therapy to the heart 12, such therapy systems may include any suitable number of leads coupled to the IMD 16, and each of the leads may extend to any location within or proximate to the heart 12. For example, other examples of therapy systems may include three transvenous leads located as illustrated in FIGS. 8-10. Still further, other therapy systems may include a single lead that extends from the IMD 16 into the right atrium 26 or the right ventricle 28, or two leads that extend into a respective one of the right atrium 26 and the right ventricle 28.

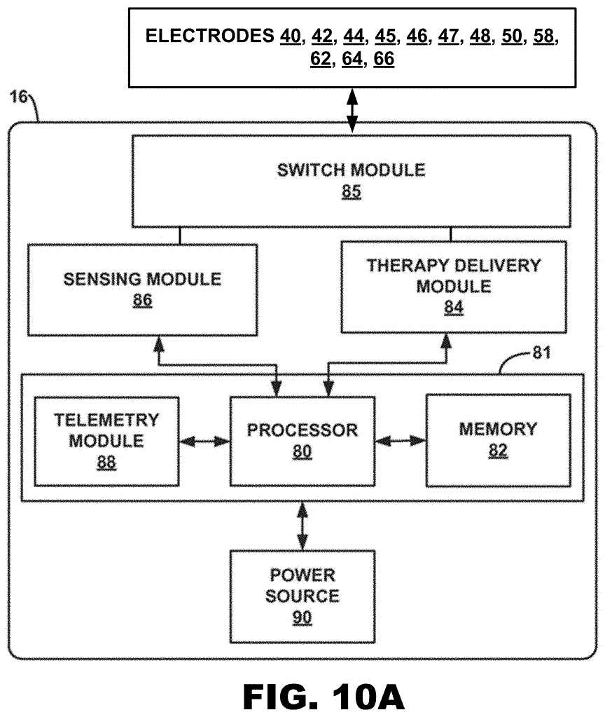

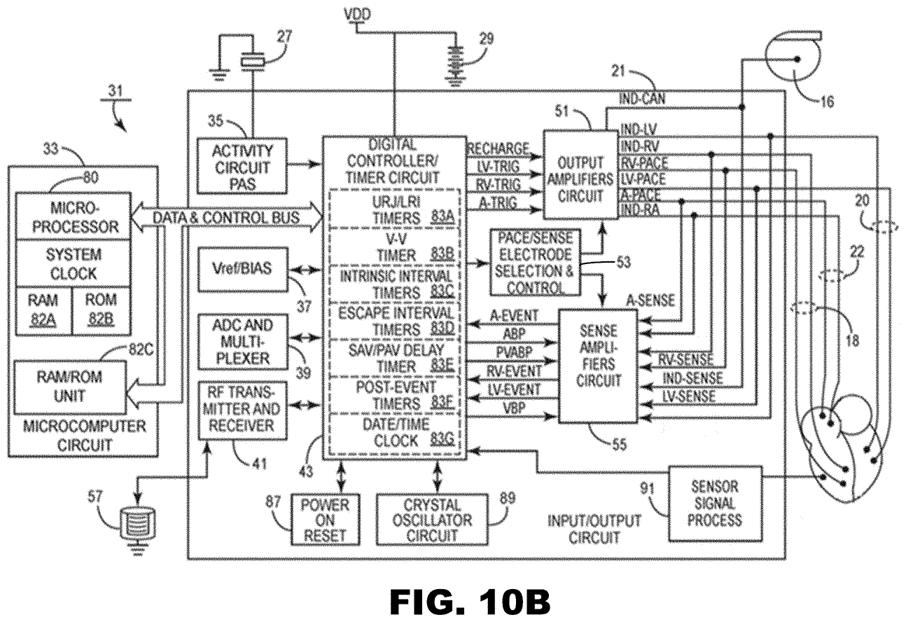

FIG. 10A is a functional block diagram of one illustrative configuration of the IMD 16. As shown, the IMD 16 may include a control module 81, a therapy delivery module 84 (e.g., which may include a stimulation generator), a sensing module 86, and a power source 90.

The control module, or apparatus, 81 may include a processor 80, memory 82, and a telemetry module, or apparatus, 88. The memory 82 may include computer-readable instructions that, when executed, e.g., by the processor 80, cause the IMD 16 and/or the control module 81 to perform various functions attributed to the IMD 16 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically-erasable programmable ROM (EEPROM), flash memory, and/or any other digital media. An illustrative capture management module may be the left ventricular capture management (LVCM) module described in U.S. Pat. No. 7,684,863 entitled “LV THRESHOLD MEASUREMENT AND CAPTURE MANAGEMENT” and issued Mar. 23, 2010, which is incorporated herein by reference in its entirety.

The processor 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the processor 80 may include multiple components, such as any combination of one or more microprocessors, one or more controllers, one or more DSPs, one or more ASICs, and/or one or more FPGAs, as well as other discrete or integrated logic circuitry. The functions attributed to the processor 80 herein may be embodied as software, firmware, hardware, or any combination thereof.

The control module 81 may control the therapy delivery module 84 to deliver therapy (e.g., electrical stimulation therapy such as pacing) to the heart 12 according to a selected one or more therapy programs, which may be stored in the memory 82. More, specifically, the control module 81 (e.g., the processor 80) may control various parameters of the electrical stimulus delivered by the therapy delivery module 84 such as, e.g., A-V delays, V-V delays, pacing pulses with the amplitudes, pulse widths, frequency, or electrode polarities, etc., which may be specified by one or more selected therapy programs (e.g., A-V and/or V-V delay adjustment programs, pacing therapy programs, pacing recovery programs, capture management programs, etc.). As shown, the therapy delivery module 84 is electrically coupled to electrodes 40, 42, 44, 45, 46, 47, 48, 50, 58, 62, 64, 66, e.g., via conductors of the respective lead 18, 20, 22, or, in the case of housing electrode 58, via an electrical conductor disposed within housing 60 of IMD 16. Therapy delivery module 84 may be configured to generate and deliver electrical stimulation therapy such as pacing therapy to the heart 12 using one or more of the electrodes 40, 42, 44, 45, 46, 47, 48, 50, 58, 62, 64, 66.

For example, therapy delivery module 84 may deliver pacing stimulus (e.g., pacing pulses) via ring electrodes 40, 44, 45, 46, 47, 48 coupled to leads 18, 20, 22 and/or helical tip electrodes 42, 50 of leads 18, 22. Further, for example, therapy delivery module 84 may deliver defibrillation shocks to heart 12 via at least two of electrodes 58, 62, 64, 66. In some examples, therapy delivery module 84 may be configured to deliver pacing, cardioversion, or defibrillation stimulation in the form of electrical pulses. In other examples, therapy delivery module 84 may be configured deliver one or more of these types of stimulation in the form of other signals, such as sine waves, square waves, and/or other substantially continuous time signals.

The IMD 16 may further include a switch module 85 and the control module 81 (e.g., the processor 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module 86 and/or the therapy delivery module 84 to one or more selected electrodes. More specifically, the therapy delivery module 84 may include a plurality of pacing output circuits. Each pacing output circuit of the plurality of pacing output circuits may be selectively coupled, e.g., using the switch module 85, to one or more of the electrodes 40, 42, 44, 45, 46, 47, 48, 50, 58, 62, 64, 66 (e.g., a pair of electrodes for delivery of therapy to a bipolar or multipolar pacing vector). In other words, each electrode can be selectively coupled to one of the pacing output circuits of the therapy delivery module using the switching module 85.

The sensing module 86 is coupled (e.g., electrically coupled) to sensing apparatus, which may include, among additional sensing apparatus, the electrodes 40, 42, 44, 45, 46, 47, 48, 50, 58, 62, 64, 66 to monitor electrical activity of the heart 12, e.g., electrocardiogram (ECG)/electrogram (EGM) signals, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave alternans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, ST segment (i.e., the segment that connects the QRS complex and the T-wave), T-wave changes, QT intervals, electrical vectors, etc.

The switch module 85 may also be used with the sensing module 86 to select which of the available electrodes are used, or enabled, to, e.g., sense electrical activity of the patient's heart (e.g., one or more electrical vectors of the patient's heart using any combination of the electrodes 40, 42, 44, 45, 46, 47, 48, 50, 58, 62, 64, 66). Likewise, the switch module 85 may also be used with the sensing module 86 to select which of the available electrodes are not to be used (e.g., disabled) to, e.g., sense electrical activity of the patient's heart (e.g., one or more electrical vectors of the patient's heart using any combination of the electrodes 40, 42, 44, 45, 46, 47, 48, 50, 58, 62, 64, 66), etc. In some examples, the control module 81 may select the electrodes that function as sensing electrodes via the switch module within the sensing module 86, e.g., by providing signals via a data/address bus.

In some examples, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In some examples, the storage of such EGMs in memory 82 may be under the control of a direct memory access circuit.

In some examples, the control module 81 may operate as an interrupt-driven device and may be responsive to interrupts from pacer timing and control module, where the interrupts may correspond to the occurrences of sensed P-waves and R-waves and the generation of cardiac pacing pulses. Any necessary mathematical calculations may be performed by the processor 80 and any updating of the values or intervals controlled by the pacer timing and control module may take place following such interrupts. A portion of memory 82 may be configured as a plurality of recirculating buffers, capable of holding one or more series of measured intervals, which may be analyzed by, e.g., the processor 80 in response to the occurrence of a pace or sense interrupt to determine whether the patient's heart 12 is presently exhibiting atrial or ventricular tachyarrhythmia.

The telemetry module 88 of the control module 81 may include any suitable hardware, firmware, software, or any combination thereof for communicating with another device, such as a programmer. For example, under the control of the processor 80, the telemetry module 88 may receive downlink telemetry from and send uplink telemetry to a programmer with the aid of an antenna, which may be internal and/or external. The processor 80 may provide the data to be uplinked to a programmer and the control signals for the telemetry circuit within the telemetry module 88, e.g., via an address/data bus. In some examples, the telemetry module 88 may provide received data to the processor 80 via a multiplexer.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

FIG. 10B is another embodiment of a functional block diagram for IMD 16 that depicts bipolar RA lead 22, bipolar RV lead 18, and bipolar LV CS lead 20 without the LA CS pace/sense electrodes and coupled with an implantable pulse generator (IPG) circuit 31 having programmable modes and parameters of a bi-ventricular DDD/R type known in the pacing art. In turn, the sensor signal processing circuit 91 indirectly couples to the timing circuit 43 and via data and control bus to microcomputer circuitry 33. The IPG circuit 31 is illustrated in a functional block diagram divided generally into a microcomputer circuit 33 and a pacing circuit 21. The pacing circuit 21 includes the digital controller/timer circuit 43, the output amplifiers circuit 51, the sense amplifiers circuit 55, the RF telemetry transceiver 41, the activity sensor circuit 35 as well as a number of other circuits and components described below.

Crystal oscillator circuit 89 provides the basic timing clock for the pacing circuit 21 while battery 29 provides power. Power-on-reset circuit 87 responds to initial connection of the circuit to the battery for defining an initial operating condition and similarly, resets the operative state of the device in response to detection of a low battery condition. Reference mode circuit 37 generates stable voltage reference and currents for the analog circuits within the pacing circuit 21. Analog-to-digital converter (ADC) and multiplexer circuit 39 digitize analog signals and voltage to provide, e.g., real time telemetry of cardiac signals from sense amplifiers 55 for uplink transmission via RF transmitter and receiver circuit 41. Voltage reference and bias circuit 37, ADC and multiplexer 39, power-on-reset circuit 87, and crystal oscillator circuit 89 may correspond to any of those used in illustrative implantable cardiac pacemakers.

If the IPG is programmed to a rate responsive mode, the signals output by one or more physiologic sensors are employed as a rate control parameter (RCP) to derive a physiologic escape interval. For example, the escape interval is adjusted proportionally to the patient's activity level developed in the patient activity sensor (PAS) circuit 35 in the depicted, illustrative IPG circuit 31. The patient activity sensor 27 is coupled to the IPG housing and may take the form of a piezoelectric crystal transducer. The output signal of the patient activity sensor 27 may be processed and used as an RCP. Sensor 27 generates electrical signals in response to sensed physical activity that are processed by activity circuit 35 and provided to digital controller/timer circuit 43. Activity circuit 35 and associated sensor 27 may correspond to the circuitry disclosed in U.S. Pat. No. 5,052,388 entitled “METHOD AND APPARATUS FOR IMPLEMENTING ACTIVITY SENSING IN A PULSE GENERATOR” and issued on Oct. 1, 1991 and U.S. Pat. No. 4,428,378 entitled “RATE ADAPTIVE PACER” and issued on Jan. 31, 1984, each of which is incorporated herein by reference in its entirety. Similarly, the illustrative systems, apparatus, and methods described herein may be practiced in conjunction with alternate types of sensors such as oxygenation sensors, pressure sensors, pH sensors, and respiration sensors, for use in providing rate responsive pacing capabilities. Alternately, QT time may be used as a rate indicating parameter, in which case no extra sensor is required. Similarly, the illustrative embodiments described herein may also be practiced in non-rate responsive pacemakers.

Data transmission to and from the external programmer is accomplished by way of the telemetry antenna 57 and an associated RF transceiver 41, which serves both to demodulate received downlink telemetry and to transmit uplink telemetry. Uplink telemetry capabilities may include the ability to transmit stored digital information, e.g., operating modes and parameters, EGM histograms, and other events, as well as real time EGMs of atrial and/or ventricular electrical activity and marker channel pulses indicating the occurrence of sensed and paced depolarizations in the atrium and ventricle.

Microcomputer 33 contains a microprocessor 80 and associated system clock and on-processor RAM and ROM chips 82A and 82B, respectively. In addition, microcomputer circuit 33 includes a separate RAM/ROM chip 82C to provide additional memory capacity. Microprocessor 80 normally operates in a reduced power consumption mode and is interrupt driven. Microprocessor 80 is awakened in response to defined interrupt events, which may include A-TRIG, RV-TRIG, LV-TRIG signals generated by timers in digital timer/controller circuit 43 and A-EVENT, RV-EVENT, and LV-EVENT signals generated by sense amplifiers circuit 55, among others. The specific values of the intervals and delays timed out by digital controller/timer circuit 43 are controlled by the microcomputer circuit 33 by way of data and control bus from programmed-in parameter values and operating modes. In addition, if programmed to operate as a rate responsive pacemaker, a timed interrupt, e.g., every cycle or every two seconds, may be provided in order to allow the microprocessor to analyze the activity sensor data and update the basic A-A, V-A, or V-V escape interval, as applicable. In addition, the microprocessor 80 may also serve to define variable, operative A-V delay intervals, V-V delay intervals, and the energy delivered to each ventricle and/or atrium.

In one embodiment, microprocessor 80 is a custom microprocessor adapted to fetch and execute instructions stored in RAM/ROM unit 82 in a conventional manner. It is contemplated, however, that other implementations may be suitable to practice the present disclosure. For example, an off-the-shelf, commercially available microprocessor or microcontroller, or custom application-specific, hardwired logic, or state-machine type circuit may perform the functions of microprocessor 80.

Digital controller/timer circuit 43 operates under the general control of the microcomputer 33 to control timing and other functions within the pacing circuit 21 and includes a set of timing and associated logic circuits of which certain ones pertinent to the present disclosure are depicted. The depicted timing circuits include URI/LRI timers 83A, V-V delay timer 83B, intrinsic interval timers 83C for timing elapsed V-EVENT to V-EVENT intervals or V-EVENT to A-EVENT intervals or the V-V conduction interval, escape interval timers 83D for timing A-A, V-A, and/or V-V pacing escape intervals, an A-V delay interval timer 83E for timing the A-LVp delay (or A-RVp delay) from a preceding A-EVENT or A-TRIG, a post-ventricular timer 83F for timing post-ventricular time periods, and a date/time clock 83G.

The A-V delay interval timer 83E is loaded with an appropriate delay interval for one ventricular chamber (e.g., either an A-RVp delay or an A-LVp) to time-out starting from a preceding A-PACE or A-EVENT. The interval timer 83E triggers pacing stimulus delivery and can be based on one or more prior cardiac cycles (or from a data set empirically derived for a given patient).

The post-event timer 83F times out the post-ventricular time period following an RV-EVENT or LV-EVENT or a RV-TRIG or LV-TRIG and post-atrial time periods following an A-EVENT or A-TRIG. The durations of the post-event time periods may also be selected as programmable parameters stored in the microcomputer 33. The post-ventricular time periods include the PVARP, a post-atrial ventricular blanking period (PAVBP), a ventricular blanking period (VBP), a post-ventricular atrial blanking period (PVARP) and a ventricular refractory period (VRP) although other periods can be suitably defined depending, at least in part, on the operative circuitry employed in the pacing engine. The post-atrial time periods include an atrial refractory period (ARP) during which an A-EVENT is ignored for the purpose of resetting any A-V delay, and an atrial blanking period (ABP) during which atrial sensing is disabled. It should be noted that the starting of the post-atrial time periods and the A-V delays can be commenced substantially simultaneously with the start or end of each A-EVENT or A-TRIG or, in the latter case, upon the end of the A-PACE which may follow the A-TRIG. Similarly, the starting of the post-ventricular time periods and the V-A escape interval can be commenced substantially simultaneously with the start or end of the V-EVENT or V-TRIG or, in the latter case, upon the end of the V-PACE which may follow the V-TRIG. The microprocessor 80 also optionally calculates A-V delays, V-V delays, post-ventricular time periods, and post-atrial time periods that vary with the sensor-based escape interval established in response to the RCP(s) and/or with the intrinsic atrial and/or ventricular rate.

The output amplifiers circuit 51 contains a RA pace pulse generator (and a LA pace pulse generator if LA pacing is provided), a RV pace pulse generator, a LV pace pulse generator, and/or any other pulse generator configured to provide atrial and ventricular pacing. In order to trigger generation of an RV-PACE or LV-PACE pulse, digital controller/timer circuit 43 generates the RV-TRIG signal at the time-out of the A-RVp delay (in the case of RV pre-excitation) or the LV-TRIG at the time-out of the A-LVp delay (in the case of LV pre-excitation) provided by A-V delay interval timer 83E (or the V-V delay timer 83B). Similarly, digital controller/timer circuit 43 generates an RA-TRIG signal that triggers output of an RA-PACE pulse (or an LA-TRIG signal that triggers output of an LA-PACE pulse, if provided) at the end of the V-A escape interval timed by escape interval timers 83D.

The output amplifiers circuit 51 includes switching circuits for coupling selected pace electrode pairs from among the lead conductors and the IND-CAN electrode 20 to the RA pace pulse generator (and LA pace pulse generator if provided), RV pace pulse generator and LV pace pulse generator. Pace/sense electrode pair selection and control circuit 53 selects lead conductors and associated pace electrode pairs to be coupled with the atrial and ventricular output amplifiers within output amplifiers circuit 51 for accomplishing RA, LA, RV and LV pacing.

The sense amplifiers circuit 55 contains sense amplifiers for atrial and ventricular pacing and sensing. High impedance P-wave and R-wave sense amplifiers may be used to amplify a voltage difference signal that is generated across the sense electrode pairs by the passage of cardiac depolarization wavefronts. The high impedance sense amplifiers use high gain to amplify the low amplitude signals and rely on pass band filters, time domain filtering and amplitude threshold comparison to discriminate a P-wave or R-wave from background electrical noise. Digital controller/timer circuit 43 controls sensitivity settings of the atrial and ventricular sense amplifiers 55.

The sense amplifiers may be uncoupled from the sense electrodes during the blanking periods before, during, and after delivery of a pace pulse to any of the pace electrodes of the pacing system to avoid saturation of the sense amplifiers. The sense amplifiers circuit 55 includes blanking circuits for uncoupling the selected pairs of the lead conductors and the IND-CAN electrode 20 from the inputs of the RA sense amplifier (and LA sense amplifier if provided), RV sense amplifier and LV sense amplifier during the ABP, PVABP and VBP. The sense amplifiers circuit 55 also includes switching circuits for coupling selected sense electrode lead conductors and the IND-CAN electrode 20 to the RA sense amplifier (and LA sense amplifier if provided), RV sense amplifier and LV sense amplifier. Again, sense electrode selection and control circuit 53 selects conductors and associated sense electrode pairs to be coupled with the atrial and ventricular sense amplifiers within the output amplifiers circuit 51 and sense amplifiers circuit 55 for accomplishing RA, LA, RV, and LV sensing along desired unipolar and bipolar sensing vectors.

Right atrial depolarizations or P-waves in the RA-SENSE signal that are sensed by the RA sense amplifier result in a RA-EVENT signal that is communicated to the digital controller/timer circuit 43. Similarly, left atrial depolarizations or P-waves in the LA-SENSE signal that are sensed by the LA sense amplifier, if provided, result in a LA-EVENT signal that is communicated to the digital controller/timer circuit 43. Ventricular depolarizations or R-waves in the RV-SENSE signal are sensed by a ventricular sense amplifier result in an RV-EVENT signal that is communicated to the digital controller/timer circuit 43. Similarly, ventricular depolarizations or R-waves in the LV-SENSE signal are sensed by a ventricular sense amplifier result in an LV-EVENT signal that is communicated to the digital controller/timer circuit 43. The RV-EVENT, LV-EVENT, and RA-EVENT, LA-SENSE signals may be refractory or non-refractory and can inadvertently be triggered by electrical noise signals or aberrantly conducted depolarization waves rather than true R-waves or P-waves.

The techniques described in this disclosure, including those attributed to the IMD 16, the remote computing apparatus 140, and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

### Illustrative Embodiments

Embodiment 1: A system for use with a remote computing apparatus to noninvasively evaluate a cardiac condition of a patient comprising:

electrode apparatus comprising a plurality of external electrodes to monitor electrical activity from tissue of a patient;

a local computing apparatus comprising processing circuitry and a communication interface, the local computing apparatus operably coupled to the electrode apparatus, the local computing apparatus configured to:

monitor, using the electrode apparatus, electrical activity from the tissue of the patient;

transmit, using the communication interface, the monitored electrical activity or data related to the monitored electrical activity to a remote computing apparatus; and

receive, using the communication interface, an indication of the cardiac condition of the patient from the remote computing apparatus in response to the transmission of the monitored electrical activity or data related to the monitored electrical activity.

Embodiment 2: The system as set forth in embodiment 1 further comprising the remote computing apparatus, the remote computing apparatus comprising:

a remote communication interface; and

processing circuitry, wherein the remote computing apparatus configured to:

receive, using the remote communication interface, the transmitted monitored electrical activity or data related to the monitored electrical activity from the local computing apparatus;

determine the indication of the cardiac condition of the patient based on the received monitored electrical activity or data related to the monitored electrical activity; and

transmit, using the remote communication interface, the indication of the cardiac condition of the patient to the local computing apparatus.

Embodiment 3: The system as set forth in embodiment 2, wherein the remote computing apparatus is further configured to:

wherein determining the indication of the cardiac condition of the patient based on the received monitored electrical activity or data related to the monitored electrical activity comprises:

tracking the transmitted monitored electrical activity or data related to the monitored electrical activity from the local computing apparatus over a time period, and

determining a trend in the transmitted monitored electrical activity or data related to the monitored electrical activity over the time period.

Embodiment 4: The system as set forth in any one of embodiments 1-3, wherein the local computing apparatus comprises a smartphone.

Embodiment 5: The system as set forth in any one of embodiments 1-4, wherein the local computing apparatus is further configured to generate electrical heterogeneity information (EHI) based on the monitored electrical activity, wherein the data related to the monitored electrical activity comprises the EHI.

Embodiment 6: The system as set forth in embodiment 5, wherein the EHI comprises a standard deviation of electrical activation times monitored by the plurality of external electrodes.

Embodiment 7: The system as set forth in embodiment 5, wherein the plurality of electrodes comprises two or more left external electrodes located proximate the left side of the patient, wherein the EHI comprises an average of electrical activation times monitored by the two or more left external electrodes.

Embodiment 8: The system as set forth in any one of embodiments 1-7, wherein the electrical activity comprises electrical activation times representative of depolarization of cardiac tissue that propagates through the torso of the patient.

Embodiment 9: The system as set forth in any one of embodiments 1-8, wherein the plurality of external electrodes comprises a plurality of surface electrodes to be located proximate skin of the patient's torso.

Embodiment 10: A method for use with a remote system to noninvasively evaluate a cardiac condition of a patient comprising:

monitoring electrical activity from the tissue of the patient using a plurality of external electrodes;

transmitting the monitored electrical activity or data related to the monitored electrical activity to a remote computing apparatus; and

receiving an indication of the cardiac condition of the patient from the remote computing apparatus in response to the transmission of the monitored electrical activity or data related to the monitored electrical activity.

Embodiment 11: The method as set forth in embodiment 10, further comprising:

receiving the transmitted monitored electrical activity or data related to the monitored electrical activity;

determining the indication of the cardiac condition of the patient based on the received monitored electrical activity or data related to the monitored electrical activity; and

transmitting the indication of the cardiac condition of the patient.

Embodiment 12: The method as set forth in embodiment 11, wherein determining the indication of the cardiac condition of the patient based on the received monitored electrical activity or data related to the monitored electrical activity comprises:

tracking the transmitted monitored electrical activity or data related to the monitored electrical activity from the local computing apparatus over a time period, and

determining a trend in the transmitted monitored electrical activity or data related to the monitored electrical activity over the time period.

Embodiment 13: The method as set forth in any one of embodiments 11-12, further comprising generating electrical heterogeneity information (EHI) based on the monitored electrical activity, wherein the data related to the monitored electrical activity comprises the EHI.

Embodiment 14: The method as set forth in embodiment 13, wherein the EHI comprises a standard deviation of electrical activation times monitored by the plurality of external electrodes.

Embodiment 15: The method as set forth in embodiment 13, wherein the plurality of electrodes comprises two or more left external electrodes located proximate the left side of the patient, wherein the EHI comprises an average of electrical activation times monitored by the two or more left external electrodes.

Embodiment 16: The method as set forth in any one of embodiments 11-15, wherein the electrical activity comprises electrical activation times representative of depolarization of cardiac tissue that propagates through the torso of the patient.

Embodiment 17: The method as set forth in any one of embodiments 11-16, wherein the plurality of external electrodes comprises a plurality of surface electrodes to be located proximate skin of the patient's torso.

Embodiment 18: A system to noninvasively evaluate a cardiac condition of a patient comprising:

electrode apparatus comprising a plurality of external electrodes to monitor electrical activity from tissue of a patient; and

a computing apparatus comprising processing circuitry and operably coupled to the electrode apparatus, the computing apparatus configured to:

measure, using the electrode apparatus, electrical activity from the tissue of the patient during a plurality of sessions, and

determine an indication of the cardiac condition of the patient based on the electrical activity over the plurality of sessions.

Embodiment 19: The system as set forth in embodiment 18, wherein measuring, using the electrode apparatus, electrical activity from the tissue of the patient during a plurality of sessions comprises measuring intrinsic electrical activity from the tissue of the patient for each of the plurality of sessions.

Embodiment 20: The system as set forth in any one of embodiments 18-19, wherein a duration between sessions is greater than or equal to 12 hours.

Embodiment 21: The system as set forth in any one of embodiments 18-19, wherein the computing apparatus is further configured to generate electrical heterogeneity information (EHI) for each session based on the measured electrical activity, wherein determining an indication of the cardiac condition of the patient based on the electrical activity over the plurality of sessions comprises determining the indication of the cardiac condition of the patient based on the EHI over the plurality of sessions.

Embodiment 22: The system as set forth in embodiment 21, wherein the EHI comprises a standard deviation of electrical activation times monitored by the plurality of external electrodes.

Embodiment 23: The system as set forth in embodiment 22, wherein determining the indication of the cardiac condition of the patient based on the EHI over the plurality of sessions comprises determining the patient would benefit from cardiac therapy if the standard deviation of electrical activation times for a session is greater than or equal to a selected threshold or if the standard deviation of electrical activation times for a session has increased by a selected percentage from the initial session.

Embodiment 24: The system as set forth in embodiment 21, wherein the plurality of electrodes comprises two or more left external electrodes located proximate the left side of the patient, wherein the EHI comprises a left average of electrical activation times monitored by the two or more left external electrodes.

Embodiment 25: The system as set forth in embodiment 24, wherein determining the indication of the cardiac condition of the patient based on the EHI over the plurality of sessions comprises determining the patient would benefit from cardiac therapy if the left average of electrical activation times for a session is greater than or equal to a selected threshold or if the left average of electrical activation times monitored for a session has changed a selected percentage from the initial session.

Embodiment 26: The system as set forth in any one of embodiments 18-25, wherein the electrical activity comprises electrical activation times representative of depolarization of cardiac tissue that propagates through the torso of the patient.

Embodiment 27: The system as set forth in any one of embodiments 18-26, wherein the plurality of external electrodes comprises a plurality of surface electrodes to be located proximate skin of the patient's torso.

Embodiment 28: The system as set forth in any one of embodiments 18-27, where the computing apparatus further comprises a communication interfaced and is further configured to transmit an alert, using the communication interface, to a remote system if the indication of the cardiac condition of the patient indicates that the patient would likely benefit from cardiac therapy.

Embodiment 29: A method to noninvasively evaluate a cardiac condition of a patient comprising:

measuring, using a plurality of external electrodes, electrical activity from the tissue of the patient during a plurality of sessions; and

determining an indication of the cardiac condition of the patient based on the electrical activity over the plurality of sessions.

Embodiment 30: The method as set forth in embodiment 29, wherein measuring electrical activity from the tissue of the patient during a plurality of sessions comprises measuring intrinsic electrical activity from the tissue of the patient for each of the plurality of sessions.

Embodiment 31: The method as set forth in any one of embodiments 29-30, wherein a duration between sessions is greater than or equal to 12 hours.

Embodiment 32: The method as set forth in any one of embodiments 29-31, the method further comprising generating electrical heterogeneity information (EHI) for each session based on the measured electrical activity, wherein determining an indication of the cardiac condition of the patient based on the electrical activity over the plurality of sessions comprises determining the indication of the cardiac condition of the patient based on the EHI over the plurality of sessions.

Embodiment 33: The method as set forth in embodiment 32, wherein the EHI comprises a standard deviation of electrical activation times monitored by the plurality of external electrodes.

Embodiment 34: The method as set forth in embodiment 32, wherein the plurality of electrodes comprises two or more left external electrodes located proximate the left side of the patient, wherein the EHI comprises a left average of electrical activation times monitored by the two or more left external electrodes.

Embodiment 35: The method as set forth in any one of embodiments 29-34, where the method further comprises transmitting an alert, using the communication interface, to a remote system if the indication of the cardiac condition of the patient indicates that the patient would likely benefit from cardiac therapy.

Embodiment 36: The method as set forth in any one of embodiments 29-35, wherein the electrical activity comprises electrical activation times representative of depolarization of cardiac tissue that propagates through the torso of the patient.

Embodiment 37: The method as set forth in any one of embodiments 29-35, wherein the plurality of external electrodes comprises a plurality of surface electrodes to be located proximate skin of the patient's torso.

This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

What is claimed:

1. A system for use with a remote computing apparatus to noninvasively evaluate a cardiac condition of a patient comprising:
electrode apparatus comprising a plurality of external electrodes to monitor electrical activity from tissue of a patient;
a local computing apparatus comprising processing circuitry and a communication interface, the local computing apparatus operably coupled to the electrode apparatus, the local computing apparatus configured to:
monitor, using the electrode apparatus, electrical activity from the tissue of the patient;
transmit, using the communication interface, the monitored electrical activity or data related to the monitored electrical activity to a remote computing apparatus; and
receive, using the communication interface, an indication of the cardiac condition of the patient from the remote computing apparatus in response to the transmission of the monitored electrical activity or data related to the monitored electrical activity.

2. The system of claim 1 further comprising the remote computing apparatus, the remote computing apparatus comprising:
a remote communication interface; and
processing circuitry, wherein the remote computing apparatus configured to:
receive, using the remote communication interface, the transmitted monitored electrical activity or data related to the monitored electrical activity from the local computing apparatus;
determine the indication of the cardiac condition of the patient based on the received monitored electrical activity or data related to the monitored electrical activity; and
transmit, using the remote communication interface, the indication of the cardiac condition of the patient to the local computing apparatus.

3. The system of claim 2, wherein the remote computing apparatus is further configured to:
wherein determining the indication of the cardiac condition of the patient based on the received monitored electrical activity or data related to the monitored electrical activity comprises:
tracking the transmitted monitored electrical activity or data related to the monitored electrical activity from the local computing apparatus over a time period, and
determining a trend in the transmitted monitored electrical activity or data related to the monitored electrical activity over the time period.

4. The system of claim 1, wherein the local computing apparatus comprises a smartphone.

5. The system of claim 1, wherein the local computing apparatus is further configured to generate electrical heterogeneity information (EHI) based on the monitored electrical activity, wherein the data related to the monitored electrical activity comprises the EHI.

6. The system of claim 5, wherein the EHI comprises a standard deviation of electrical activation times monitored by the plurality of external electrodes.

7. The system of claim 5, wherein the plurality of electrodes comprises two or more left external electrodes located proximate the left side of the patient, wherein the EHI comprises an average of electrical activation times monitored by the two or more left external electrodes.

8. The system of claim 1, wherein the electrical activity comprises electrical activation times representative of depolarization of cardiac tissue that propagates through the torso of the patient.

9. The system of claim 1, wherein the plurality of external electrodes comprises a plurality of surface electrodes to be located proximate skin of the patient's torso.

10. A method for use with a remote system to noninvasively evaluate a cardiac condition of a patient comprising:
monitoring electrical activity from the tissue of the patient using a plurality of external electrodes;
transmitting the monitored electrical activity or data related to the monitored electrical activity to a remote computing apparatus; and
receiving an indication of the cardiac condition of the patient from the remote computing apparatus in response to the transmission of the monitored electrical activity or data related to the monitored electrical activity.

11. The method of claim 10, further comprising:
receiving the transmitted monitored electrical activity or data related to the monitored electrical activity;
determining the indication of the cardiac condition of the patient based on the received monitored electrical activity or data related to the monitored electrical activity; and
transmitting the indication of the cardiac condition of the patient.

12. The method of claim 11, wherein determining the indication of the cardiac condition of the patient based on the received monitored electrical activity or data related to the monitored electrical activity comprises:
tracking the transmitted monitored electrical activity or data related to the monitored electrical activity from the local computing apparatus over a time period, and
determining a trend in the transmitted monitored electrical activity or data related to the monitored electrical activity over the time period.

13. The method of claim 11, further comprising generating electrical heterogeneity information (EHI) based on the monitored electrical activity, wherein the data related to the monitored electrical activity comprises the EHI.

14. The method of claim 13, wherein the EHI comprises a standard deviation of electrical activation times monitored by the plurality of external electrodes.

15. The method of claim 11, wherein the plurality of electrodes comprises two or more left external electrodes located proximate the left side of the patient, wherein the EHI comprises an average of electrical activation times monitored by the two or more left external electrodes.

16. A system to noninvasively evaluate a cardiac condition of a patient comprising:
electrode apparatus comprising a plurality of external electrodes to monitor electrical activity from tissue of a patient; and
a computing apparatus comprising processing circuitry and operably coupled to the electrode apparatus, the computing apparatus configured to:
measure, using the electrode apparatus, electrical activity from the tissue of the patient during a plurality of sessions, and
determine an indication of the cardiac condition of the patient based on the electrical activity over the plurality of sessions.

17. The system of claim 16, wherein measuring, using the electrode apparatus, electrical activity from the tissue of the patient during a plurality of sessions comprises measuring intrinsic electrical activity from the tissue of the patient for each of the plurality of sessions.

18. The system of claim 16, wherein a duration between sessions is greater than or equal to 12 hours.

19. The system of claim 16, wherein the computing apparatus is further configured to generate electrical heterogeneity information (EHI) for each session based on the measured electrical activity, wherein determining an indication of the cardiac condition of the patient based on the electrical activity over the plurality of sessions comprises determining the indication of the cardiac condition of the patient based on the EHI over the plurality of sessions.

20. The system of claim 19, wherein the EHI comprises a standard deviation of electrical activation times monitored by the plurality of external electrodes.

21. The system of claim 20, wherein determining the indication of the cardiac condition of the patient based on the EHI over the plurality of sessions comprises determining the patient would benefit from cardiac therapy if the standard deviation of electrical activation times for a session is greater than or equal to a selected threshold or if the standard deviation of electrical activation times for a session has increased by a selected percentage from the initial session.

22. The system of claim 19, wherein the plurality of electrodes comprises two or more left external electrodes located proximate the left side of the patient, wherein the EHI comprises a left average of electrical activation times monitored by the two or more left external electrodes.

23. The system of claim 22, wherein determining the indication of the cardiac condition of the patient based on the EHI over the plurality of sessions comprises determining the patient would benefit from cardiac therapy if the left average of electrical activation times for a session is greater than or equal to a selected threshold or if the left average of electrical activation times monitored for a session has changed a selected percentage from the initial session.

24. The system of claim 16, wherein the electrical activity comprises electrical activation times representative of depolarization of cardiac tissue that propagates through the torso of the patient.

25. The system of claim 16, wherein the plurality of external electrodes comprises a plurality of surface electrodes to be located proximate skin of the patient's torso.

26. The system of claim 16, where the computing apparatus further comprises a communication interfaced and is further configured to transmit an alert, using the communication interface, to a remote system if the indication of the cardiac condition of the patient indicates that the patient would likely benefit from cardiac therapy.

27. A method to noninvasively evaluate a cardiac condition of a patient comprising:
measuring, using a plurality of external electrodes, electrical activity from the tissue of the patient during a plurality of sessions; and
determining an indication of the cardiac condition of the patient based on the electrical activity over the plurality of sessions.

28. The method of claim 27, wherein measuring electrical activity from the tissue of the patient during a plurality of sessions comprises measuring intrinsic electrical activity from the tissue of the patient for each of the plurality of sessions.

29. The method of claim 27, wherein a duration between sessions is greater than or equal to 12 hours.

30. The method of claim 27, the method further comprising generating electrical heterogeneity information (EHI) for each session based on the measured electrical activity, wherein determining an indication of the cardiac condition of the patient based on the electrical activity over the plurality of sessions comprises determining the indication of the cardiac condition of the patient based on the EHI over the plurality of sessions.

31. The method of claim 30, wherein the EHI comprises a standard deviation of electrical activation times monitored by the plurality of external electrodes.

32. The method of claim 30, wherein the plurality of electrodes comprises two or more left external electrodes located proximate the left side of the patient, wherein the EHI comprises a left average of electrical activation times monitored by the two or more left external electrodes.

33. The method of claim 27, where the method further comprises transmitting an alert, using the communication interface, to a remote system if the indication of the cardiac condition of the patient indicates that the patient would likely benefit from cardiac therapy.
